SEQUENCE LISTING (18 sequences)

SEQ ID NO: 1 - DNA, 40 bp, Homo sapiens
tgggaggctccgacccacagatgcagccccaccccacgga

SEQ ID NO: 2 - DNA, 40 bp, Homo sapiens
gggaagacctgctggcagaggaggagcccccatccccacg

SEQ ID NO: 3 - DNA, 40 bp, Homo sapiens
gggaagacctgctggcagagacctcctgccccctggcggc

SEQ ID NO: 4 - DNA, 6759 bp, Homo sapiens
atggacgaggaggaggatggagcgggcgccgaggagtcgggacagccccggagcttcatgcggctcaacgacctgtcgggggccgggggccggccggggccggggtcagcagaaaaggacccgggcagcgcggactccgaggcggaggggctgccgtacccggcgctggccccggtggttttcttctacttgagccaggacagccgcccgcggagctggtgtctccgcacggtctgtaacccctggtttgagcgcatcagcatgttggtcatccttctcaactgcgtgaccctgggcatgttccggccatgcgaggacatcgcctgtgactcccagcgctgccggatcctgcaggcctttgatgacttcatctttgccttctttgccgtggagatggtggtgaagatggtggccttgggcatctttgggaaaaagtgttacctgggagacacttggaaccggcttgactttttcatcgtcatcgcagggatgctggagtactcgctggacctgcagaacgtcagcttctcagctgtcaggacagtccgtgtgctgcgaccgctcagggccattaaccgggtgcccagcatgcgcatccttgtcacgttgctgctggatacgctgcccatgctgggcaacgtcctgctgctctgcttcttcgtcttcttcatcttcggcatcgtcggcgtccagctgtgggcagggctgcttcggaaccgatgcttcctacctgagaatttcagcctccccctgagcgtggacctggagcgctattaccagacagagaacgaggatgagagccccttcatctgctcccagccacgcgagaacggcatgcggtcctgcagaagcgtgcccacgctgcgcggggacgggggcggtggcccaccttgcggtctggactatgaggcctacaacagctccagcaacaccacctgtgtcaactggaaccagtactacaccaactgctcagcgggggagcacaaccccttcaagggcgccatcaactttgacaacattggctatgcctggatcgccatcttccaggtcatcacgctggagggctgggtcgacatcatgtactttgtgatggatgctcattccttctacaatttcatctacttcatcctcctcatcatcgtgggctccttcttcatgatcaacctgtgcctggtggtgattgccacgcagttctcagagaccaagcagcgggaaagccagctgatgcgggagcagcgtgtgcggttcctgtccaacgccagcaccctggctagcttctctgagcccggcagctgctatgaggagctgctcaagtacctggtgtacatccttcgtaaggcagcccgcaggctggctcaggtctctcgggcagcaggtgtgcgggttgggctgctcagcagcccagcacccctcgggggccaggagacccagcccagcagcagctgctctcgctcccaccgccgcctatccgtccaccacctggtgcaccaccaccaccaccatcaccaccactaccacctgggcaatgggacgctcagggccccccgggccagcccggagatccaggacagggatgccaatgggtcccgccggctcatgctgccaccaccctcgacgcctgccctctccggggccccccctggtggcgcagagtctgtgcacagcttctaccatgccgactgccacttagagccagtccgctgccaggcgccccctcccaggtccccatctgaggcatccggcaggactgtgggcagcgggaaggtgtatcccaccgtgcacaccagccctccaccggagacgctgaaggagaaggcactagtagaggtggctgccagctctgggcccccaaccctcaccagcctcaacatcccacccgggccctacagctccatgcacaagctgctggagacacagagtacaggtgcctgccaaagctcttgcaagatctccagcccttgcttgaaagcagacagtggagcctgtggtccagacagctgcccctactgtgcccgggccggggcaggggaggtggagctcgccgaccgtgaaatgcctgactcagacagcgaggcagtttatgagttcacacaggatgcccagcacagcgacctccgggacccccacagccggcggcaacggagcctgggcccagatgcagagcccagctctgtgctggccttctggaggctaatctgtgacaccttccgaaagattgtggacagcaagtactttggccggggaatcatgatcgccatcctggtcaacacactcagcatgggcatcgaataccacgagcagcccgaggagcttaccaacgccctagaaatcagcaacatcgtcttcaccagcctctttgccctggagatgctgctgaagctgcttgtgtatggtccctttggctacatcaagaatccctacaacatcttcgatggtgtcattgtggtcatcagcgtgtgggagatcgtgggccagcaggggggcggcctgtcggtgctgcggaccttccgcctgatgcgtgtgctgaagctggtgcgcttcctgccggcgctgcagcggcagctggtggtgctcatgaagaccatggacaacgtggccaccttctgcatgctgcttatgctcttcatcttcatcttcagcatcctgggcatgcatctcttcggctgcaagtttgcctctgagcgggatggggacaccctgccagaccggaagaattttgactccttgctctgggccatcgtcactgtctttcagatcctgacccaggaggactggaacaaagtcctctacaatggtatggcctccacgtcgtcctgggcggccctttatttcattgccctcatgaccttcggcaactacgtgctcttcaatttgctggtcgccattctggtggagggcttccaggcggaggaaatcagcaaacgggaagatgcgagtggacagttaagctgtattcagctgcctgtcgactcccaggggggagatgccaacaagtccgaatcagagcccgatttcttctcacccagcctggatggtgatggggacaggaagaagtgcttggccttggtgtccctgggagagcacccggagctgcggaagagcctgctgccgcctctcatcatccacacggccgccacacccatgtcgctgcccaagagcaccagcacgggcctgggcgaggcgctgggccctgcgtcgcgccgcaccagcagcagcgggtcggcagagcctggggcggcccacgagatgaagtcaccgcccagcgcccgcagctctccgcacagcccctggagcgctgcaagcagctggaccagcaggcgctccagccggaacagcctcggccgtgcacccagcctgaagcggagaagcccaagtggagagcggcggtccctgttgtcgggagaaggccaggagagccaggatgaagaggagagctcagaagaggagcgggccagccctgcgggcagtgaccatcgccacagggggtccctggagcgggaggccaagagttcctttgacctgccagacacactgcaggtgccagggctgcatcgcactgccagtggccgagggtctgcttctgagcaccaggactgcaatggcaagtcggcttcagggcgcctggcccgggccctgcggcctgatgaccccccactggatggggatgacgccgatgacgagggcaacctgagcaaaggggaacgggtccgcgcgtggatccgagcccgactccctgcctgctgcctcgagcgagactcctggtcagcctacatcttccctcctcagtccaggttccgcctcctgtgtcaccggatcatcacccacaagatgttcgaccacgtggtccttgtcatcatcttccttaactgcatcaccatcgccatggagcgccccaaaattgacccccacagcgctgaacgcatcttcctgaccctctccaattacatcttcaccgcagtctttctggctgaaatgacagtgaaggtggtggcactgggctggtgcttcggggagcaggcgtacctgcggagcagttggaacgtgctggacgggctgttggtgctcatctccgtcatcgacattctggtgtccatggtctctgacagcggcaccaagatcctgggcatgctgagggtgctgcggctgctgcggaccctgcgcccgctcagggtgatcagccgggcgcaggggctgaagctggtggtggagacgctgatgtcctcactgaaacccatcggcaacattgtagtcatctgctgtgccttcttcatcattttcggcatcttgggggtgcagctcttcaaagggaagtttttcgtgtgccagggcgaggataccaggaacatcaccaataaatcggactgtgccgaggccagttaccggtgggtccggcacaagtacaactttgacaaccttggccaggccctgatgtccctgttcgttttggcctccaaggatggttgggtggacatcatgtacgatgggctggatgctgtgggcgtggaccagcagcccatcatgaaccacaacccctggatgctgctgtacttcatctcgttcctgctcattgtggccttctttgtcctgaacatgtttgtgggtgtggtggtggagaacttccacaagtgtcggcagcaccaggaggaagaggaggcccggcggcgggaggagaagcgcctacgaagactggagaaaaagagaaggaatctaatgctggacgatgtaattgcttccggcagctcagccagcgctgcgtcagaagcccagtgcaaaccttactactccgactactcccgcttccggctcctcgtccaccacttgtgcaccagccactacctggacctcttcatcacaggtgtcatcgggctgaacgtggtcaccatggccatggagcactaccagcagccccagattctggatgaggctctgaagatctgcaactacatcttcactgtcatctttgtcttggagtcagttttcaaacttgtggcctttggtttccgtcggttcttccaggacaggtggaaccagctggacctggccattgtgctgctgtccatcatgggcatcacgctggaggaaatcgaggtcaacgcctcgctgcccatcaaccccaccatcatccgcatcatgagggtgctgcgcattgcccgagtgctgaagctgctgaagatggctgtgggcatgcgggcgctgctggacacggtgatgcaggccctgccccaggtggggaacctgggacttctcttcatgttgttgtttttcatctttgcagctctgggcgtggagctctttggagacctggagtgtgacgagacacacccctgtgagggcctgggccgtcatgccacctttcggaactttggcatggccttcctaaccctcttccgagtctccacaggtgacaattggaatggcattatgaaggacaccctccgggactgtgaccaggagtccacctgctacaacacggtcatctcgcctatctactttgtgtccttcgtgctgacggcccagttcgtgctagtcaacgtggtgatcgccgtgctgatgaagcacctggaggagagcaacaaggaggccaaggaggaggccgagctagaggctgagctggagctggagatgaagaccctcagcccccagccccactcgccactgggcagccccttcctctggcctggggtcgagggccccgacagccccgacagccccaagcctggggctctgcacccagcggcccacgcgagatcagcctcccacttttccctggagcaccccacggacaggcagctgtttgacaccatatccctgctgatccagggctccctggagtgggagctgaagctgatggacgagctggcaggcccagggggccagccctctgccttcccttctgcccccagcctgggaggctccgacccacagatgcagccccaccccacggagctgccaggaccagacttactgactgtgcggaagtctggggtcagccgaacgcactctctgcccaatgacagctacatgtgtcggcatgggagcactgccgaggggcccctgggacacaggggctgggggctccccaaagctcagtcaggctccgtcttgtccgttcactcccagccagcagataccagctacatcctgcagcttcccaaagatgcacctcatctgctccagccccacagcgccccaacctggggcaccatccccaaactgcccccaccaggacgctcccctttggctcagaggccactcaggcgccaggcagcaataaggactgactccttggacgttcagggtctgggcagccgggaagacctgctggcagaggaggagcccccatccccacgggacctgaagaagtgctacagcgtggaggcccagagctgccagcgccggcctacgtcctggctggatgagcagaggagacactctatcgccgtcagctgcctggacagcggctcccaaccccacctgggcacagacccctctaaccttgggggccagcctcttggggggcctgggagccggcccaagaaaaaactcagcccgcctagtatcaccatagacccccccgagagccaaggtcctcggaccccgcccagccctggtatctgcctccggaggagggctccgtccagcgactccaaggatcccttggcctctggcccccctgacagcatggctgcctcgccctccccaaagaaagatgtgctgagtctctccggtttatcctctgacccagcagacctggacccc

SEQ ID NO: 5 - protein, 2253 aa, Homo sapiens
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEEISKREDASGQLSCIQLPVDSQGGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRNLMLDDVIASGSSASAASEAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGPDSPDSPKPGALHPAAHARSASHFSLEHPTDRQLFDTISLLIQGSLEWELKLMDELAGPGGQPSAFPSAPSLGGSDPQMQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAEEEPPSPRDLKKCYSVEAQSCQRRPTSWLDEQRRHSIAVSCLDSGSQPHLGTDPSNLGGQPLGGPGSRPKKKLSPPSITIDPPESQGPRTPPSPGICLRRRAPSSDSKDPLASGPPDSMAASPSPKKDVLSLSGLSSDPADLDP

SEQ ID NO: 6 - DNA, 6378 bp, Homo sapiens
atggacgaggaggaggatggagcgggcgccgaggagtcgggacagccccggagcttcatgcggctcaacgacctgtcgggggccgggggccggccggggccggggtcagcagaaaaggacccgggcagcgcggactccgaggcggaggggctgccgtacccggcgctggccccggtggttttcttctacttgagccaggacagccgcccgcggagctggtgtctccgcacggtctgtaacccctggtttgagcgcatcagcatgttggtcatccttctcaactgcgtgaccctgggcatgttccggccatgcgaggacatcgcctgtgactcccagcgctgccggatcctgcaggcctttgatgacttcatctttgccttctttgccgtggagatggtggtgaagatggtggccttgggcatctttgggaaaaagtgttacctgggagacacttggaaccggcttgactttttcatcgtcatcgcagggatgctggagtactcgctggacctgcagaacgtcagcttctcagctgtcaggacagtccgtgtgctgcgaccgctcagggccattaaccgggtgcccagcatgcgcatccttgtcacgttgctgctggatacgctgcccatgctgggcaacgtcctgctgctctgcttcttcgtcttcttcatcttcggcatcgtcggcgtccagctgtgggcagggctgcttcggaaccgatgcttcctacctgagaatttcagcctccccctgagcgtggacctggagcgctattaccagacagagaacgaggatgagagccccttcatctgctcccagccacgcgagaacggcatgcggtcctgcagaagcgtgcccacgctgcgcggggacgggggcggtggcccaccttgcggtctggactatgaggcctacaacagctccagcaacaccacctgtgtcaactggaaccagtactacaccaactgctcagcgggggagcacaaccccttcaagggcgccatcaactttgacaacattggctatgcctggatcgccatcttccaggtcatcacgctggagggctgggtcgacatcatgtactttgtgatggatgctcattccttctacaatttcatctacttcatcctcctcatcatcgtgggctccttcttcatgatcaacctgtgcctggtggtgattgccacgcagttctcagagaccaagcagcgggaaagccagctgatgcgggagcagcgtgtgcggttcctgtccaacgccagcaccctggctagcttctctgagcccggcagctgctatgaggagctgctcaagtacctggtgtacatccttcgtaaggcagcccgcaggctggctcaggtctctcgggcagcaggtgtgcgggttgggctgctcagcagcccagcacccctcgggggccaggagacccagcccagcagcagctgctctcgctcccaccgccgcctatccgtccaccacctggtgcaccaccaccaccaccatcaccaccactaccacctgggcaatgggacgctcagggccccccgggccagcccggagatccaggacagggatgccaatgggtcccgccggctcatgctgccaccaccctcgacgcctgccctctccggggccccccctggtggcgcagagtctgtgcacagcttctaccatgccgactgccacttagagccagtccgctgccaggcgccccctcccaggtccccatctgaggcatccggcaggactgtgggcagcgggaaggtgtatcccaccgtgcacaccagccctccaccggagacgctgaaggagaaggcactagtagaggtggctgccagctctgggcccccaaccctcaccagcctcaacatcccacccgggccctacagctccatgcacaagctgctggagacacagagtacaggtgcctgccaaagctcttgcaagatctccagcccttgcttgaaagcagacagtggagcctgtggtccagacagctgcccctactgtgcccgggccggggcaggggaggtggagctcgccgaccgtgaaatgcctgactcagacagcgaggcagtttatgagttcacacaggatgcccagcacagcgacctccgggacccccacagccggcggcaacggagcctgggcccagatgcagagcccagctctgtgctggccttctggaggctaatctgtgacaccttccgaaagattgtggacagcaagtactttggccggggaatcatgatcgccatcctggtcaacacactcagcatgggcatcgaataccacgagcagcccgaggagcttaccaacgccctagaaatcagcaacatcgtcttcaccagcctctttgccctggagatgctgctgaagctgcttgtgtatggtccctttggctacatcaagaatccctacaacatcttcgatggtgtcattgtggtcatcagcgtgtgggagatcgtgggccagcaggggggcggcctgtcggtgctgcggaccttccgcctgatgcgtgtgctgaagctggtgcgcttcctgccggcgctgcagcggcagctggtggtgctcatgaagaccatggacaacgtggccaccttctgcatgctgcttatgctcttcatcttcatcttcagcatcctgggcatgcatctcttcggctgcaagtttgcctctgagcgggatggggacaccctgccagaccggaagaattttgactccttgctctgggccatcgtcactgtctttcagatcctgacccaggaggactggaacaaagtcctctacaatggtatggcctccacgtcgtcctgggcggccctttatttcattgccctcatgaccttcggcaactacgtgctcttcaatttgctggtcgccattctggtggagggcttccaggcggaggaaatcagcaaacgggaagatgcgagtggacagttaagctgtattcagctgcctgtcgactcccaggggggagatgccaacaagtccgaatcagagcccgatttcttctcacccagcctggatggtgatggggacaggaagaagtgcttggccttggtgtccctgggagagcacccggagctgcggaagagcctgctgccgcctctcatcatccacacggccgccacacccatgtcgctgcccaagagcaccagcacgggcctgggcgaggcgctgggccctgcgtcgcgccgcaccagcagcagcgggtcggcagagcctggggcggcccacgagatgaagtcaccgcccagcgcccgcagctctccgcacagcccctggagcgctgcaagcagctggaccagcaggcgctccagccggaacagcctcggccgtgcacccagcctgaagcggagaagcccaagtggagagcggcggtccctgttgtcgggagaaggccaggagagccaggatgaagaggagagctcagaagaggagcgggccagccctgcgggcagtgaccatcgccacagggggtccctggagcgggaggccaagagttcctttgacctgccagacacactgcaggtgccagggctgcatcgcactgccagtggccgagggtctgcttctgagcaccaggactgcaatggcaagtcggcttcagggcgcctggcccgggccctgcggcctgatgaccccccactggatggggatgacgccgatgacgagggcaacctgagcaaaggggaacgggtccgcgcgtggatccgagcccgactccctgcctgctgcctcgagcgagactcctggtcagcctacatcttccctcctcagtccaggttccgcctcctgtgtcaccggatcatcacccacaagatgttcgaccacgtggtccttgtcatcatcttccttaactgcatcaccatcgccatggagcgccccaaaattgacccccacagcgctgaacgcatcttcctgaccctctccaattacatcttcaccgcagtctttctggctgaaatgacagtgaaggtggtggcactgggctggtgcttcggggagcaggcgtacctgcggagcagttggaacgtgctggacgggctgttggtgctcatctccgtcatcgacattctggtgtccatggtctctgacagcggcaccaagatcctgggcatgctgagggtgctgcggctgctgcggaccctgcgcccgctcagggtgatcagccgggcgcaggggctgaagctggtggtggagacgctgatgtcctcactgaaacccatcggcaacattgtagtcatctgctgtgccttcttcatcattttcggcatcttgggggtgcagctcttcaaagggaagtttttcgtgtgccagggcgaggataccaggaacatcaccaataaatcggactgtgccgaggccagttaccggtgggtccggcacaagtacaactttgacaaccttggccaggccctgatgtccctgttcgttttggcctccaaggatggttgggtggacatcatgtacgatgggctggatgctgtgggcgtggaccagcagcccatcatgaaccacaacccctggatgctgctgtacttcatctcgttcctgctcattgtggccttctttgtcctgaacatgtttgtgggtgtggtggtggagaacttccacaagtgtcggcagcaccaggaggaagaggaggcccggcggcgggaggagaagcgcctacgaagactggagaaaaagagaaggaatctaatgctggacgatgtaattgcttccggcagctcagccagcgctgcgtcagaagcccagtgcaaaccttactactccgactactcccgcttccggctcctcgtccaccacttgtgcaccagccactacctggacctcttcatcacaggtgtcatcgggctgaacgtggtcaccatggccatggagcactaccagcagccccagattctggatgaggctctgaagatctgcaactacatcttcactgtcatctttgtcttggagtcagttttcaaacttgtggcctttggtttccgtcggttcttccaggacaggtggaaccagctggacctggccattgtgctgctgtccatcatgggcatcacgctggaggaaatcgaggtcaacgcctcgctgcccatcaaccccaccatcatccgcatcatgagggtgctgcgcattgcccgagtgctgaagctgctgaagatggctgtgggcatgcgggcgctgctggacacggtgatgcaggccctgccccaggtggggaacctgggacttctcttcatgttgttgtttttcatctttgcagctctgggcgtggagctctttggagacctggagtgtgacgagacacacccctgtgagggcctgggccgtcatgccacctttcggaactttggcatggccttcctaaccctcttccgagtctccacaggtgacaattggaatggcattatgaaggacaccctccgggactgtgaccaggagtccacctgctacaacacggtcatctcgcctatctactttgtgtccttcgtgctgacggcccagttcgtgctagtcaacgtggtgatcgccgtgctgatgaagcacctggaggagagcaacaaggaggccaaggaggaggccgagctagaggctgagctggagctggagatgaagaccctcagcccccagccccactcgccactgggcagccccttcctctggcctggggtcgagggccccgacagccccgacagccccaagcctggggctctgcacccagcggcccacgcgagatcagcctcccacttttccctggagcaccccacggacaggcagctgtttgacaccatatccctgctgatccagggctccctggagtgggagctgaagctgatggacgagctggcaggcccagggggccagccctctgccttcccttctgcccccagcctgggaggctccgacccacagatgcagccccaccccacggagctgccaggaccagacttactgactgtgcggaagtctggggtcagccgaacgcactctctgcccaatgacagctacatgtgtcggcatgggagcactgccgaggggcccctgggacacaggggctgggggctccccaaagctcagtcaggctccgtcttgtccgttcactcccagccagcagataccagctacatcctgcagcttcccaaagatgcacctcatctgctccagccccacagcgccccaacctggggcaccatccccaaactgcccccaccaggacgctcccctttggctcagaggccactcaggcgccaggcagcaataaggactgactccttggacgttcagggtctgggcagccgggaagacctgctggcagagacctcctgccccctggcggccaggaggagcccccatccccacgggacc

SEQ ID NO: 7 - protein, 2126 aa, Homo sapiens
MDEEEDGAGAEESGQPRSFMRLNDLSGAGGRPGPGSAEKDPGSADSEAEGLPYPALAPVVFFYLSQDSRPRSWCLRTVCNPWFERISMLVILLNCVTLGMFRPCEDIACDSQRCRILQAFDDFIFAFFAVEMVVKMVALGIFGKKCYLGDTWNRLDFFIVIAGMLEYSLDLQNVSFSAVRTVRVLRPLRAINRVPSMRILVTLLLDTLPMLGNVLLLCFFVFFIFGIVGVQLWAGLLRNRCFLPENFSLPLSVDLERYYQTENEDESPFICSQPRENGMRSCRSVPTLRGDGGGGPPCGLDYEAYNSSSNTTCVNWNQYYTNCSAGEHNPFKGAINFDNIGYAWIAIFQVITLEGWVDIMYFVMDAHSFYNFIYFILLIIVGSFFMINLCLVVIATQFSETKQRESQLMREQRVRFLSNASTLASFSEPGSCYEELLKYLVYILRKAARRLAQVSRAAGVRVGLLSSPAPLGGQETQPSSSCSRSHRRLSVHHLVHHHHHHHHHYHLGNGTLRAPRASPEIQDRDANGSRRLMLPPPSTPALSGAPPGGAESVHSFYHADCHLEPVRCQAPPPRSPSEASGRTVGSGKVYPTVHTSPPPETLKEKALVEVAASSGPPTLTSLNIPPGPYSSMHKLLETQSTGACQSSCKISSPCLKADSGACGPDSCPYCARAGAGEVELADREMPDSDSEAVYEFTQDAQHSDLRDPHSRRQRSLGPDAEPSSVLAFWRLICDTFRKIVDSKYFGRGIMIAILVNTLSMGIEYHEQPEELTNALEISNIVFTSLFALEMLLKLLVYGPFGYIKNPYNIFDGVIVVISVWEIVGQQGGGLSVLRTFRLMRVLKLVRFLPALQRQLVVLMKTMDNVATFCMLLMLFIFIFSILGMHLFGCKFASERDGDTLPDRKNFDSLLWAIVTVFQILTQEDWNKVLYNGMASTSSWAALYFIALMTFGNYVLFNLLVAILVEGFQAEEISKREDASGQLSCIQLPVDSQGGDANKSESEPDFFSPSLDGDGDRKKCLALVSLGEHPELRKSLLPPLIIHTAATPMSLPKSTSTGLGEALGPASRRTSSSGSAEPGAAHEMKSPPSARSSPHSPWSAASSWTSRRSSRNSLGRAPSLKRRSPSGERRSLLSGEGQESQDEEESSEEERASPAGSDHRHRGSLEREAKSSFDLPDTLQVPGLHRTASGRGSASEHQDCNGKSASGRLARALRPDDPPLDGDDADDEGNLSKGERVRAWIRARLPACCLERDSWSAYIFPPQSRFRLLCHRIITHKMFDHVVLVIIFLNCITIAMERPKIDPHSAERIFLTLSNYIFTAVFLAEMTVKVVALGWCFGEQAYLRSSWNVLDGLLVLISVIDILVSMVSDSGTKILGMLRVLRLLRTLRPLRVISRAQGLKLVVETLMSSLKPIGNIVVICCAFFIIFGILGVQLFKGKFFVCQGEDTRNITNKSDCAEASYRWVRHKYNFDNLGQALMSLFVLASKDGWVDIMYDGLDAVGVDQQPIMNHNPWMLLYFISFLLIVAFFVLNMFVGVVVENFHKCRQHQEEEEARRREEKRLRRLEKKRRNLMLDDVIASGSSASAASEAQCKPYYSDYSRFRLLVHHLCTSHYLDLFITGVIGLNVVTMAMEHYQQPQILDEALKICNYIFTVIFVLESVFKLVAFGFRRFFQDRWNQLDLAIVLLSIMGITLEEIEVNASLPINPTIIRIMRVLRIARVLKLLKMAVGMRALLDTVMQALPQVGNLGLLFMLLFFIFAALGVELFGDLECDETHPCEGLGRHATFRNFGMAFLTLFRVSTGDNWNGIMKDTLRDCDQESTCYNTVISPIYFVSFVLTAQFVLVNVVIAVLMKHLEESNKEAKEEAELEAELELEMKTLSPQPHSPLGSPFLWPGVEGPDSPDSPKPGALHPAAHARSASHFSLEHPTDRQLFDTISLLIQGSLEWELKLMDELAGPGGQPSAFPSAPSLGGSDPQMQPHPTELPGPDLLTVRKSGVSRTHSLPNDSYMCRHGSTAEGPLGHRGWGLPKAQSGSVLSVHSQPADTSYILQLPKDAPHLLQPHSAPTWGTIPKLPPPGRSPLAQRPLRRQAAIRTDSLDVQGLGSREDLLAETSCPLAARRSPHPHGT

SEQ ID NO: 8 - DNA, 66 bp, Homo sapiens
gcagcaataaggactgactccttggacgttcagggtctgggcagccgggaagacctgctggcagag

SEQ ID NO: 9 - DNA, 429 bp, Homo sapiens
gaggagcccccatccccacgggacctgaagaagtgctacagcgtggaggcccagagctgccagcgccggcctacgtcctggctggatgagcagaggagacactctatcgccgtcagctgcctggacagcggctcccaaccccacctgggcacagacccctctaaccttgggggccagcctcttggggggcctgggagccggcccaagaaaaaactcagcccgcctagtatcaccatagacccccccgagagccaaggtcctcggaccccgcccagccctggtatctgcctccggaggagggctccgtccagcgactccaaggatcccttggcctctggcccccctgacagcatggctgcctcgccctccccaaagaaagatgtgctgagtctctccggtttatcctctgacccagcagacctggacccc

SEQ ID NO: 10 - DNA, 48 bp, Homo sapiens
acctcctgccccctggcggccaggaggagcccccatccccacgggacc

SEQ ID NO: 11 - DNA, 20 bp, Homo sapiens
gctggcagaggaggagcccc

SEQ ID NO: 12 - DNA, 20 bp, Homo sapiens
gctggcagagacctcctgcc

SEQ ID NO: 13 - DNA, 20 bp, Homo sapiens
cgacccacagatgcagcccc

SEQ ID NO: 14 - protein, 10 aa, Homo sapiens
DLLAEEEPPS

SEQ ID NO: 15 - protein, 10 aa, Homo sapiens
DLLAETSCPL

SEQ ID NO: 16 - protein, 10 aa, Homo sapiens
GSDPQMQPHP

SEQ ID NO: 17 - DNA, 20 bp, Homo sapiens
gatcagcctcccacttttcc

SEQ ID NO: 18 - DNA, 20 bp, Homo sapiens
cctccacgctgtagcacttc

(19) United States
(12) Patent Application Publication
(10) Pub. No.: US 2006/0281117 A1
(43) Pub. Date: Dec. 14, 2006

(54) Alternatively spliced isoform of calcium channel, voltage dependent, alpha-1G subunit (CACNA1G)

(76) Inventors: Brian S. Roberts, Bellevue, WA (US); Christopher D. Armour, Kirkland, WA (US); Christopher K. Raymond, Seattle, WA (US)

Correspondence Address: R. Douglas Bradley;Merck & Co., Inc., P.O. Box 2000 - Patent Department RY60-30, Rahway, NJ, 07065-0907, US

(21) Appl. No.: 11/441,654

(22) Filed: May 26, 2006

Related U.S. Application Data
(60) Provisional application No. 60/689,476, filed on Jun. 10, 2005.

(51) Int. Cl. C12Q 1/68; G01N 33/567; C07H 21/04; C12P 21/06; G01N 33/53; C07K 14/705

(52) U.S. Cl. 435/6; 435/69.1; 435/320.1; 435/325; 435/7.2; 530/350; 536/23.5

(57) ABSTRACT

The present invention features nucleic acids and polypeptides encoding novel splice variant isoforms of calcium channel, voltage dependent, alpha-1G subunit (CACNA1G). The polynucleotide sequence of CACNA1Gsv1 is provided by SEQ ID NO 4. The amino acid sequence of CACNA1Gsv1 is provided by SEQ ID NO 5. The polynucleotide sequence of CACNA1Gsv2 is provided by SEQ ID NO 6. The amino acid sequence of CACNA1Gsv2 is provided by SEQ ID NO 7. The present invention also provides methods for using CACNA1Gsv1, CACNA1Gsv2 polynucleotides and proteins to screen for compounds that bind to CACNA1Gsv1, CACNA1Gsv2, respectively.

This application claims priority to U.S. Provisional Patent Application Ser. No. 60/689,476 filed on Jun. 10, 2005, which is incorporated by reference herein in its entirety.

### BACKGROUND OF THE INVENTION

Voltage-gated calcium channels mediate the influx of calcium ions in response to changes in membrane potential in electrically excitable cells such as neurons and myocytes. Calcium is an important second messenger in muscle contraction, chemotaxis, gene expression, synaptic transmission, and secretion of hormones and neurotransmitters. Its entry into the cell can also depolarize the cell membrane, activating other voltage-gated ion channels (reviewed in Perez-Reyes, 2003, Physiol. Rev. 83:117-161; Catterall, 2000, Annu. Rev. Cell Dev. Biol. 16:521-555).

To date ten genes encoding pore-forming α₁ subunits of voltage-gated calcium channels have been identified. These ten genes are grouped into three subfamilies according to their predicted amino acid sequences; these subfamily divisions also coincide with their pharmacological and biophysical properties. The Caᵥ1 subfamily consists of Caᵥ1.1-1.4 (also known as α_{1S, C, D, F}), the Caᵥ2 subfamily: Caᵥ2.1-2.3 (also known as α_{1A, B, E}), the Caᵥ3 subfamily: Caᵥ3.1-3.3 (α_{1G, H, I}). The predicted amino acid sequences of the α₁ subunits have 70% identity within a subfamily but less than 40% identity among subfamilies (Ertel et al., 2000, Neuron 25:533-535).

Caᵥ1 subfamily channels mediate L-type Ca²⁺ currents. Caᵥ2 subfamily mediates P/Q-type, N-type, and R-type currents, and the Caᵥ3 subfamily mediates T-type currents. L-type currents are long-lasting and require a strong depolarization for activation. L-type channels are blocked by organic antagonists such as dihydropyridines, phenyl alkylamines, and benzothiazepines. They are expressed in muscle and endocrine cells, where they mediate contraction and secretion. N-type, P/Q-type, and R-type calcium channels also require strong depolarization. However, they are resistant to L-type channel inhibitors but sensitive to toxins from snails and spiders. These channels are expressed in neurons, initiating neurotransmission at fast synapses. T-type channels have a low voltage threshold and have a fast time course. T-type channels are also resistant to the L-type organic antagonists and snake and spider toxins which discriminate N-, P/Q-, and R-type channels. T-type channels have been found in a wide variety of cell types, including nervous tissue, kidney, heart, smooth muscle, sperm, and endocrine organs and are implicated in neuronal firing, hormone secretion, smooth muscle contraction, myoblast fusion, and fertilization (reviewed in Catterall, 2000, Annu. Rev. Cell Dev. Biol. 16:521-555; Perez-Reyes, 2003, Physiol. Rev. 83:117-161; Ertel et al., 2000, Neuron 25:533-535).

Properties of T-type channels include: opening after small de-polarizations of the plasma membrane (low-voltage activated (LVA)); transient currents during a sustained pulse; slow closing upon membrane re-polarization, producing slow tail currents; tiny single channel conductance of Ba²⁺ and Ca²⁺; insensitivity to dihydropyridines; and similar voltage range for both activation and steady-state inactivation (reviewed in Perez-Reyes, 2003, Physiol. Rev. 83:117-161). Electrophysiological studies of recombinant CACNA1G and CACNA1H channels demonstrate that they possess similar activation and inactivation kinetics, but may be differentiated by their recovery from inactivation and sensitivity to block by Ni⁺⁺ (Klockner et al., 1999, Eur. J. Neurosci. 11:4171-4178; Lee et al., 1999, Biophys. J. 77:3034-3042; Satin et al., 2000, Circ. Res. 86:636-642). T-currents generated by CACNA1I subunits have slow activation and inactivation kinetics distinct from CACNA1G and CACNA1H (Lee et al., 1999, J. Neurosci. 19:1912-1921; Monteil et al., 2000, J. Biol. Chem. 275:16530-16535).

Calcium channels are complex proteins consisting of multiple subunits. The largest subunit, α₁, contains the conduction pore, voltage sensor, gating apparatus, and sites of channel regulation by second messengers, drugs, and toxins. The α₁ subunit has four homologous domains (Domains I to IV), each containing six α-helical transmembrane segments (S1 to S6), and a membrane-associated loop between S5 and S6 which forms the pore lining of the channel. The S4 segment serves as the voltage sensor, initiating conformational change and opening the pore upon depolarization. β, γ, and α₂δ subunits are auxiliary subunits that modulate the channel's properties (reviewed in Catterall, 2000, Annu. Rev. Cell Dev. Biol. 16:521-555). The specific subunit structures of T-type channels are unknown, as native channels have not yet been purified (reviewed in Perez-Reyes, 2003, Physiol. Rev. 83:117-161).

The rat Caᵥ3.1 gene, also known as CACNA1G, was cloned from a brain cDNA library. Homologous human and mouse EST clones were also identified and sequenced (Perez-Reyes et al., 1998, Nature 391:896-899). Human CACNA1G consists of at least 38 exons encoding a 2,377 amino acid protein and is located on chromosome 17 (Mittman et al., 1999, Neurosci. Lett. 274:143-146). Except for exons 34 and 35, the remaining exons of the human CACNA1G have counterparts in the rat or mouse cDNA sequence (Mittman et al., 1999, Neurosci. Lett. 274:143-146). Comparing the human CACNA1G to rodent sequences, the four transmembrane domains are highly conserved (98-100% identity), while the connecting loops between domains I and II and domain II and III, as well as the amino- and carboxy-terminal regions are more divergent (85-95% identity) (Monteil et al., 2000, J. Biol. Chem. 275:6090-6100). CACNA1G transcripts were detected at high levels in the human and rat brain, and less abundantly in the heart, placenta, kidney, and lung (Perez-Reyes et al., 1998, Nature 391:896-899). Human CACNA1G is only 60% identical to either human CACNA1H or CACNA1I, with the highest regions of identity, approximately 90%, being in the putative membrane spanning regions (Cribbs et al., 2000, FEBS Lett. 466:54-58).

The mouse CACNA1G sequence has also been cloned from brain. The mouse transcript encodes a 2,295 amino acid protein that and was primarily and abundantly detected in the brain (Klugbauer et al., 1999, Eur. J. Physiol. 437:710-715).

Splice variants of human CACNA1G have been described (see WO 99/29847). Jagannathan et al., (2002, J. Biol. Chem. 277:8449-8456) detected multiple isoforms of CACNA1G in human testis. Mittman et al. (Neurosci. Lett., 1999, 274:143-146) noted alternative splicing occurring at cassette exons 14, 26, 34, and 35. An internal donor in exon 25 that leads to the deletion of 21 nucleotides at the 3′ end of the exon, and a 237 nucleotide, protein-coding portion of exon 38 that could be excised as an intron were also reported. Monteil et al. (2000, J. Biol. Chem 275:6090-6100) also describes use of two possible splice donor sites in exon 25 combined with the acceptor site on exon 27 and the exclusive combination of exon 26 with the alternative “b” splice donor site in exon 25. Functional expression of a couple of these splice variants in mammalian cell lines demonstrated properties characteristic of a T-type channel (Cribbs et al., 2000, FEBS Lett. 466:54-58; Monteil et al., 2000, J. Biol. Chem. 275:6090-6100). CACNA1G splice variants have also been identified in rat insulin-secreting cells (Zhuang et al., 2000, Diabetes 49:59-64) and murine atrial myocytes (Satin and Cribbs, 2000, Circ. Res. 86:636-642). The effects of these splice variants on channel function are unclear, however, for another T-type calcium channel, CACNA1I, variations in C-terminal regions have demonstrated different current kinetics (Chemin et al., 2001, Eur. J. Neurosci. 14:1678-1686; Murbartian et al., 2002, FEBS Lett. 528:272-278; Gomora et al., 2002, Biophys. J. 83:229-241).

Few mutations in human Caᵥ3 subfamily genes have been described, but mutations in calcium channel genes have been associated with ataxic and epileptic disorders (Jen, 1999, Curr. Opin. Neurobiol. 9:274-280; Kullmann et al., 2002, Brain 125:1177-1195). The importance of T-currents in the development of absence seizures has been suggested by studies of animal models of absence seizures (Tsakiridou et al., 1995, J. Neurosci. 15:3110-3117; Zhang et al., 2002, J. Neurosci. 22:6362-6371). Study of CACNA1G knockout mice has provided evidence for the role of T-type channels in the generation of absence seizures in the thalamocortical network (Kim et al., 2001, Neuron 31:35-45). Kim et al. (2003, Science 302:117-119) also demonstrated that CACNA1G null mice show hyperalgesia to visceral pain, and thalamic infusion of a T-type channel blocker induced similar hyperalgesia in wild-type mice. Khosravani et al. (2004, J. Biol. Chem. 279:9681-9684) demonstrated that several mutations in CACNA1H associated with childhood absence epilepsy show greater calcium influx, which may increase propensity for seizures.

In contrast to high voltage-activated calcium channels, T-type channels are relatively resistant to organic calcium channel blockers and peptide toxins. While compounds that inhibit T-type channels have been identified, none of these compounds are highly selective for T-type channels (reviewed in Heady et al., 2001, Jpn. J. Pharmacol. 85:339-350). CACNA1H channels are sensitive to low concentrations of nickel, but much higher concentrations are required to half-block CACNA1G and CACNA1I (Lee et al., 1999, Biophys. J. 77:3034-3042). Mibefradil, an antihypertensive agent, has been shown to block T-type channels with a 13-fold greater affinity than the high voltage-activated L-type channel (Monteil et al., 2000, J. Biol. Chem. 275:16530-16535; Martin et al., 2000, J. Pharmacol. Exp. Ther. 295:302-308; reviewed in Heady et al., 2001, Jpn. J. Pharmacol. 85:339-350). The endogenous cannabinoid, anandamide, directly inhibits T-type channels, with CACNA1I displaying the most marked modulation compared to CACNA1G and CACNA1H (Chemin et al., 2001, EMBO J. 20:7033-7040). While kurotoxin and kurotoxin-like peptides from scorpion species inhibit T-type calcium channels, they also display cross-reactivity with voltage-gated sodium channels (Chuang et al., 1998, Nat. Neurosci. 1:668-674; Olamendi-Portugal et al., 2002, Biochem. Biophys. Res. Commun. 299:562-568). Succinimide anti-epileptic drugs are also capable of inhibiting calcium T-type channels (Gomora et al., 2001, Mol. Pharmacol. 60:1121-1132).

Calcium T-type channel function is implicated in slow wave sleep and absence epilepsy. Within thalamic neurons, T-type channels are activated by depolarization from hyperpolarized membrane potentials and generate a low threshold calcium spike, which triggers an oscillatory fire mode called burst firing. Low threshold burst firing is thought to underlie the thalamocortical rhythmic oscillations during deep sleep and absence epilepsy (reviewed in Pape et al., 2004, Pflugers Arch. 448:131-138; Perez-Reyes, 2003, Physiol. Rev. 83:117-161). In a study of the rat model of absence epilepsy, a selective increase in T-type calcium conductance of reticular thalamic neurons was observed in affected rats compared to seizure-free rats (Tsakiridou et al., 1995, J. Neurosci. 15:3110-3117). Supporting the hypothesis that T-type channels are involved in thalamocortical dysrhythmias disorders, drugs used as anti-epileptics and anesthesics demonstrate T-type channel block (reviewed in Perez-Reyes, 2003, Physiol. Rev. 83:117-161). Ethanol, which is known to disrupt normal sleep rhythms, has been found to affect calcium currents in thalamic relay cells (Mu et al., 2003, J. Pharmacol. Exp. Ther. 307:197-204). T-type calcium channel blockers have been shown to inhibit tactile and thermal hypersensitivities in a dose-dependent manner in rodent models of neuropathy, suggesting a role for T-type channels in the neuropathic state (Matthews and Dickenson, 2001, Eur. J. Pharmacol. 415:141-149; Dogrul et al., 2003, Pain 105:159-168).

Because of the multiple therapeutic values of drugs targeting calcium channels, including CACNA1G, there is a need in the art for compounds that selectively bind to isoforms of CACNA1G. The present invention is directed toward novel CACNA1G isoforms (CACNA1Gsv1, CACNA1Gsv2) and uses thereof.

### BRIEF DESCRIPTION OF THE DRAWINGS

FIG. 1A illustrates the exon structure of human CACNA1G mRNA corresponding to the known reference form of CACNA1G mRNA (labeled NM_{—}018896) and the exon structure corresponding to the inventive splice variant transcripts (labeled CACNA1Gsv1 and CACNA1Gsv2). FIG. 1B depicts the nucleotide sequences of the exon junctions resulting from the splicing of exon 34 to exon 36 in the case of CACNA1Gsv1 and CACNA1Gsv2 mRNA [SEQ ID NO 1], and the splicing of exon 35a to exon 35b in the case of CACNA1Gsv1 mRNA [SEQ ID NO 2], and the splicing of exon 35a to exon 35c in the case of CACNA1Gsv2 mRNA [SEQ ID NO 3]. In FIG. 1B, in the case of the CACNA1Gsv1 and CACNA1Gsv2 exon 34-exon 36 splice junction sequence [SEQ ID NO 1], the nucleotides shown in italics represent the 20 nucleotides at the 3′ end of exon 34 and the nucleotides shown in underline represent the 20 nucleotides at the 5′ end of exon 36; and in the case of CACNA1Gsv1 exon 38a-exon 38b splice junction sequence [SEQ ID NO 2], the nucleotides shown in italics represent the 20 nucleotides at the 3′ end of exon 38a and the nucleotides shown in underline represent the 20 nucleotides at the 5′ end of exon 38b; and in the case of CACNA1Gsv2 exon 38a-exon 38c splice junction sequence [SEQ ID NO 3], the nucleotides shown in italics represent the 20 nucleotides at the 3′ end of exon 38a and the nucleotides shown in underline represent the 20 nucleotides at the 5′ end of exon 38c.

### SUMMARY OF THE INVENTION

RT-PCR and DNA sequence analysis have been used to identify and confirm the presence of novel splice variants of human CACNA1G mRNA. More specifically, the present invention features polynucleotides encoding different protein isoforms of CACNA1G. A polynucleotide sequence encoding CACNA1Gsv1 is provided by SEQ ID NO 4. An amino acid sequence for CACNA1Gsv1 is provided by SEQ ID NO 5. A polynucleotide sequence encoding CACNA1Gsv2 is provided by SEQ ID NO 6. An amino acid sequence for CACNA1Gsv2 is provided by SEQ ID NO 7.

Thus, a first aspect of the present invention describes a purified CACNA1Gsv1 encoding nucleic acid and a purified CACNA1Gsv2 encoding nucleic acid. The CACNA1Gsv1 encoding nucleic acid comprises SEQ ID NO 4 or the complement thereof. The CACNA1Gsv2 encoding nucleic acid comprises SEQ ID NO 6 or the complement thereof. Reference to the presence of one region does not indicate that another region is not present. For example, in different embodiments the inventive nucleic acid can comprise, consist, or consist essentially of an encoding nucleic acid sequence of SEQ ID NO 4, or can comprise, consist, or consist essentially of the nucleic acid sequence of SEQ ID NO 6.

Another aspect of the present invention describes a purified CACNA1Gsv1 polypeptide that can comprise, consist or consist essentially of the amino acid sequence of SEQ ID NO 5. An additional aspect describes a purified CACNA1Gsv2 polypeptide that can comprise, consist or consist essentially of the amino acid sequence of SEQ ID NO 7.

Another aspect of the present invention describes expression vectors. In one embodiment of the invention, the inventive expression vector comprises a nucleotide sequence encoding a polypeptide comprising, consisting, or consisting essentially of SEQ ID NO 5, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 7, wherein the nucleotide sequence is transcriptionally coupled to an exogenous promoter.

Alternatively, the nucleotide sequence comprises, consists, or consists essentially of SEQ ID NO 4, and is transcriptionally coupled to an exogenous promoter. In another embodiment, the nucleotide sequence comprises, consists, or consists essentially or SEQ ID NO 6, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 4 or SEQ ID NO 6, or a nucleotide sequence encoding a polypeptide comprising, consisting, or consisting essentially of an amino acid sequence of SEQ ID NO 5 or SEQ ID NO 7, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing CACNA1Gsv1 or CACNA1Gsv2 polypeptide comprising SEQ ID NO 5 or SEQ ID NO 7, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to CACNA1Gsv1 as compared to one or more calcium channel isoform polypeptides that are not CACNA1Gsv1. In another embodiment, a purified antibody preparation is provided comprising antibody that binds selectively to CACNA1Gsv2 as compared to one or more calcium channel isoform polypeptides that are not CACNA1Gsv2.

Another aspect of the present invention provides a method of screening for a compound that binds to CACNA1Gsv1, CACNA1Gsv2, or fragments thereof. In one embodiment, the method comprises the steps of: (a) expressing a polypeptide comprising the amino acid sequence of SEQ ID NO 5 or a fragment thereof from recombinant nucleic acid; (b) providing to said polypeptide a labeled CACNA1G ligand that binds to said polypeptide and a test preparation comprising one or more test compounds; (c) and measuring the effect of said test preparation on binding of said test preparation to said polypeptide comprising SEQ ID NO 5. Alternatively, this method could be performed using SEQ ID NO 7, instead of SEQ ID NO 5.

In another embodiment of the method, a compound is identified that binds selectively to CACNA1Gsv1 polypeptide as compared to one or more calcium channel isoform polypeptides that are not CACNA1Gsv1. This method comprises the steps of: providing a CACNA1Gsv1 polypeptide comprising SEQ ID NO 5; providing a calcium channel isoform polypeptide that is not CACNA1Gsv1; contacting said CACNA1Gsv1 polypeptide and said calcium channel isoform polypeptide that is not CACNA1Gsv1 with a test preparation comprising one or more test compounds; and determining the binding of said test preparation to said CACNA1Gsv1 polypeptide and to said calcium channel isoform polypeptide that is not CACNA1Gsv1, wherein a test preparation that binds to said CACNA1Gsv1 polypeptide but does not bind to said calcium channel isoform polypeptide that is not CACNA1Gsv1 contains a compound that selectively binds said CACNA1Gsv1 polypeptide. Alternatively, the same method can be performed using CACNA1Gsv2 polypeptide comprising, consisting, or consisting essentially of SEQ ID NO 7.

In another embodiment of the invention, a method is provided for screening for a compound able to bind to or interact with a CACNA1Gsv1 protein or a fragment thereof comprising the steps of: expressing a CACNA1Gsv1 polypeptide comprising SEQ ID NO 5 or a fragment thereof from a recombinant nucleic acid; providing to said polypeptide a labeled CACNA1G ligand that binds to said polypeptide and a test preparation comprising one or more compounds; and measuring the effect of said test preparation on binding of said labeled CACNA1G ligand to said polypeptide, wherein a test preparation that alters the binding of said labeled CACNA1G ligand to said polypeptide contains a compound that binds to or interacts with said polypeptide. In an alternative embodiment, the method is performed using CACNA1Gsv2 polypeptide comprising, consisting, or consisting essentially of SEQ ID NO 7 or a fragment thereof.

Another aspect of the present invention provides a method of screening for a compound that binds to one or more calcium channel isoform polypeptides that are not CACNA1Gsv1. This method comprises the steps of: providing a CACNA1Gsv1 polypeptide comprising SEQ ID NO 5; providing a calcium channel isoform polypeptide that is not CACNA1Gsv1; contacting said CACNA1Gsv1 polypeptide and calcium channel isoform polypeptide that is not CACNA1Gsv1 with a test preparation comprising one or more test compounds; and determining the binding of said test preparation to said CACNA1Gsv1 polypeptide and to said calcium channel isoform polypeptide that is not CACNA1Gsv1, wherein a test preparation that binds to said calcium channel isoform polypeptide that is not CACNA1Gsv1 but not to said CACNA1Gsv1 polypeptide contains a compound that selectively binds said calcium channel isoform polypeptide. Alternatively, the same method can be performed using CACNA1Gsv2 polypeptide comprising, consisting, or consisting essentially of SEQ ID NO 7.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “CACNA1G” refers to a calcium channel, voltage gated, alpha-1G (NM_{—}018896). In contrast, reference to a CACNA1G isoform includes NM_{—}018896 and other polypeptide isoform variants of CACNA1G.

As used herein, “CACNA1Gsv1” and “CACNA1Gsv2” refer to a splice variant isoforms of human CACNA1G protein, wherein the splice variants have the amino acid sequences set forth in SEQ ID NO 5 (for CACNA1Gsv1) and SEQ ID NO 7 (for CACNA1Gsv2).

As used herein, “CACNA1G” refers to polynucleotides encoding CACNA1G.

As used herein, “CACNA1Gsv1” refers to polynucleotides that are identical to CACNA1G encoding polynucleotides, except that the sequences represented by exon 35 of the CACNA1G messenger RNA are not present and the sequences represented by an internal portion of exon 38 of the CACNA1G messenger RNA are not present in CACNA1Gsv1. “Intron 38a” refers to the polynucleotides encoding the internal portion of exon 38 that are not present in CACNA1Gsv1. “Exon 38a” and “exon 38b” refer to polynucleotides encoding the remaining portions of exon 38 retained in CACNA1Gsv1, resulting from the drop of intron 38a. As used herein, “CACNA1Gsv2” refers to polynucleotides that are identical to CACNA1G encoding polynucleotides, except that the sequences represented by exon 35 of the CACNA1G messenger RNA are not present and the sequences represented by an internal portion of exon 38 of the CACNA1G messenger RNA are not present in CACNA1Gsv2. “Intron 38b” refers to the polynucleotides encoding the internal portion of exon 38 that are not present in CACNA1Gsv2. “Exon 38a” and “exon 38c” refer to polynucleotides encoding the remaining portions of exon 38 retained in CACNA1Gsv2, resulting from the drop of intron 38b. The polynucleotide sequence of exon 38a is set forth in SEQ ID NO 8. The polynucleotide sequence of exon 38b is set forth in SEQ ID NO 9. The polynucleotides sequence of exon 38c is set forth in SEQ ID NO 10.

As used herein, “CACNA1Gsv1” refers to polynucleotides encoding CACNA1Gsv1 having an amino acid sequence set forth in SEQ ID NO 5. As used herein, “CACNA1Gsv2” refers to polynucleotides encoding CACNA1Gsv2 having an amino acid sequence set forth in SEQ ID NO 7.

As used herein, a “calcium channel isoform” is any isoform of any calcium channel from any organism, including but not limited to human CACNA1S (Caᵥ1.1), CACNA1C (Caᵥ1.2), CACNA1D (Caᵥ1.3), CACNA1A (Caᵥ2.1), CACNA1B (Caᵥ2.2), CACNA1E (Caᵥ2.3), CACNA1G (Caᵥ3.1), CACNA1H (Caᵥ3.2), and CACNA1I (Caᵥ3.3).

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is non-identical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequence encoding human CACNA1Gsv1 and CACNA1Gsv2 that are alternatively spliced isoforms of CACNA1G, and to the amino acid sequences encoding these proteins. SEQ ID NO 4 and SEQ ID NO 6 are polynucleotide sequences representing an exemplary open reading frame that encode the CACNA1Gsv1 and CACNA1Gsv2 proteins, respectively. SEQ ID NO 5 shows the polypeptide sequence of CACNA1Gsv1. SEQ ID NO 7 shows the polypeptide sequence of CACNA1Gsv2.

CACNA1Gsv1 and CACNA1Gsv2 polynucleotide sequences encoding CACNA1Gsv1 and CACNA1Gsv2 proteins, as exemplified and enabled herein include a number of specific, substantial and credible utilities. For example, CACNA1Gsv1 and CACNA1Gsv2 encoding nucleic acids were identified in an mRNA sample obtained from a human source (see Example 1). Such nucleic acids can be used as hybridization probes to distinguish between cells that produce CACNA1Gsv1 and CACNA1Gsv2 transcripts from human or non-human cells (including bacteria) that do not produce such transcripts. Similarly, antibodies specific for CACNA1Gsv1 or CACNA1Gsv2 can be used to distinguish between cells that express CACNA1Gsv1 or CACNA1Gsv2 from human or non-human cells (including bacteria) that do not express CACNA1Gsv1 or CACNA1Gsv2.

The importance of CACNA1G as a drug target for disorders featuring thalamocortical dysrhythmias including absence epilepsy and sleep disorders, is evidenced by the selective increase in T-type calcium conductance of reticular thalamic neurons in a genetic absence epilepsy rat model and the channel blocking effects of antiepileptic and anesthetic drugs (Tsakiridou et al., J. Neurosci. 1995, 15:3110-3117; Perez-Reyes, 2003, Physiol. Rev. 83:117-161). CACNA1G function is also implicated in an antinocioceptive mechanism, as demonstrated with CACNA1G null mice (Kim et al., 2003, Science 302:117-119). Given the potential importance of CACNA1G activity to the therapeutic management of epilepsy or sleep disorders and neuropathic pain, it is of value to identify CACNA1G isoforms and identify CACNA1G-ligand compounds that are isoform specific, as well as compounds that are effective ligands for two or more different CACNA1G isoforms or calcium channel isoforms. In particular, it may be important to identify compounds that are effective inhibitors of a specific CACNA1G isoform activity, yet do not bind to or interact with a plurality of different CACNA1G isoforms or calcium channel isoforms. Compounds that bind to or interact with multiple CACNA1G isoforms may require higher drug doses to saturate multiple CACNA1G-isoform binding sites and thereby result in a greater likelihood of secondary non-therapeutic side effects. Furthermore, biological effects could also be caused by the interaction of a drug with the CACNA1Gsv1 or CACNA1GSv2 isoforms specifically. For the foregoing reasons, CACNA1Gsv1 and CACNA1Gsv2 proteins represent useful compound binding targets and have utility in the identification of new CACNA1G-ligands and calcium channel isoform-ligands exhibiting a preferred specificity profile and having greater efficacy for their intended use.

In some embodiments, CACNA1Gsv1 and CACNA1Gsv2 activity is modulated by a ligand compound to achieve one or more of the following: prevent or reduce the risk of occurrence, or recurrence of neuropathic pain and thalamocortical dysrhythmia disorders including epilepsy and sleep disorders.

Compounds modulating CACNA1Gsv1 or CACNA1Gsv2 include agonists, antagonists, and allosteric modulators. While not wishing to be limited to any particular theory of therapeutic efficacy, generally, but not always, CACNA1Gsv1 or CACNA1Gsv2 compounds will be used to modulate the activity of the CACNA1G voltage gated calcium channel. These compounds may act as pore blockers and inhibit the passage of calcium ions across the cellular membrane (Gomora et al., 2001, Mol. Pharmacol. 60:1121-1132). Compounds that bind and stabilize T-channels in the inactivated state may also be utilized (Chemin et al., 2001, EMBO J. 20:7033-7040). Peptides that interact with the voltage-sensing domain and modify channel gating may be used a channel blockers (Chuang et al., 1998, Nature Neurosci. 1:668-674). Calcium channel blocking drugs have been used as anti-arrhythmic drugs and anti-convulsants (reviewed in Heady et al., 2001, Jpn. J. Pharmacol. 85:339-350). Therefore, agents that modulate CACNA1G activity may be used to achieve a therapeutic benefit for any disease or condition due to, or exacerbated by, CACNA1G ion channel activity.

CACNA1Gsv1 or CACNA1Gsv2 activity can also be affected by modulating the cellular abundance of transcripts encoding CACNA1Gsv1 or CACNA1Gsv2, respectively. Compounds modulating the abundance of transcripts encoding CACNA1Gsv1 or CACNA1Gsv2 include a cloned polynucleotide encoding CACNA1Gsv1 or CACNA1Gsv2, respectively, that can express CACNA1Gsv1 or CACNA1Gsv2 in vivo, antisense nucleic acids targeted to CACNA1Gsv1 or CACNA1Gsv2 transcripts, enzymatic nucleic acids, such as ribozymes, and RNAi nucleic acids, such as shRNAs or siRNAs, targeted to CACNA1Gsv1 or CACNA1Gsv2 transcripts.

In some embodiments, CACNA1Gsv1 or CACNA1Gsv2 activity is modulated to achieve a therapeutic effect upon diseases in which regulation of CACNA1G is desirable. For example, neuropathic pain, epilepsy and sleep disorders may be treated by modulating CACNA1Gsv1 or CACNA1Gsv2 calcium channel activity.

CACNA1Gsv1 and CACNA1Gsv2 Nucleic Acids

CACNA1Gsv1 nucleic acids contain regions that encode for polypeptides comprising, consisting, or consisting essentially of SEQ ID NO 5. CACNA1Gsv2 nucleic acids contain regions that encode for polypeptides comprising, consisting, or consisting essentially of SEQ ID NO 7. The CACNA1Gsv1 and CACNA1Gsv2 nucleic acids have a variety of uses, such as use as a hybridization probe or PCR primer to identify the presence of CACNA1Gsv1 or CACNA1Gsv2 nucleic acids, respectively; use as a hybridization probe or PCR primer to identify nucleic acids encoding for proteins related to CACNA1Gsv1 or CACNA1Gsv2, respectively; and/or use for recombinant expression of CACNA1Gsv1 or CACNA1Gsv2 polypeptides. In particular, CACNA1Gsv1 polynucleotides do not have the polynucleotide regions that consist of exon 35 and a portion of exon 38, referred to as intron 38a, of the CACNA1G gene. CACNA1Gsv2 polynucleotides do not have the polynucleotide regions that consist of exon 35 and a portion of exon 38, referred to as intron 38b, of the CACNA1G gene.

The invention also encompasses a polymorphic variant of a nucleotide sequence encoding CACNA1Gsv1 or CACNA1Gsv2. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

Regions in CACNA1Gsv1 or CACNA1Gsv2 nucleic acid that do not encode for CACNA1Gsv1 or CACNA1Gsv2, or are not found in SEQ ID NO 4, or SEQ ID NO 6, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding CACNA1Gsv1 or CACNA1Gsv2 related proteins from different sources. Obtaining nucleic acids encoding CACNA1Gsv1 or CACNA1Gsv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

CACNA1Gsv1 or CACNA1Gsv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A = Ala = Alanine: codons GCA, GCC, GCG, GCU
C = Cys = Cysteine: codons UGC, UGU
D = Asp = Aspartic acid: codons GAC, GAU
E = Glu = Glutamic acid: codons GAA, GAG
F = Phe = Phenylalanine: codons UUC, UUU
G = Gly = Glycine: codons GGA, GGC, GGG, GGU
H = His = Histidine: codons CAC, CAU
I = Ile = Isoleucine: codons AUA, AUC, AUU
K = Lys = Lysine: codons AAA, AAG
L = Leu = Leucine: codons UUA, UUG, CUA, CUC, CUG, CUU
M = Met = Methionine: codon AUG
N = Asn = Asparagine: codons AAC, AAU
P = Pro = Proline: codons CCA, CCC, CCG, CCU
Q = Gln = Glutamine: codons CAA, GAG
R = Arg = Arginine: codons AGA, AGG, CGA, CGC, CGG, CGU
S = Ser = Serine: codons AGC, AGU, UCA, UCC, UCG, UCU
T = Thr = Threonine: codons ACA, ACC, ACG, ACU
V = Val = Valine: codons GUA, GUC, GUG, GUU
W = Trp = Tryptophan: codon UGG
Y = Tyr = Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

CACNA1Gsv1 and CACNA1Gsv2 Probes

Probes for CACNA1Gsv1 or CACNA1Gsv2 contain a region that can specifically hybridize to CACNA1Gsv1 or CACNA1Gsv2 target nucleic acids, respectively, under appropriate hybridization conditions and can distinguish CACNA1Gsv1 or CACNA1Gsv2 nucleic acids from each other and from non-target nucleic acids, in particular CACNA1G polynucleotides containing exon 35 and intron 38a and CACNA1G polynucleotides containing exon 35 and intron 38b. Probes for CACNA1Gsv1 or CACNA1Gsv2 can also contain nucleic acid regions that are not complementary to CACNA1Gsv1 or CACNA1Gsv2 nucleic acids.

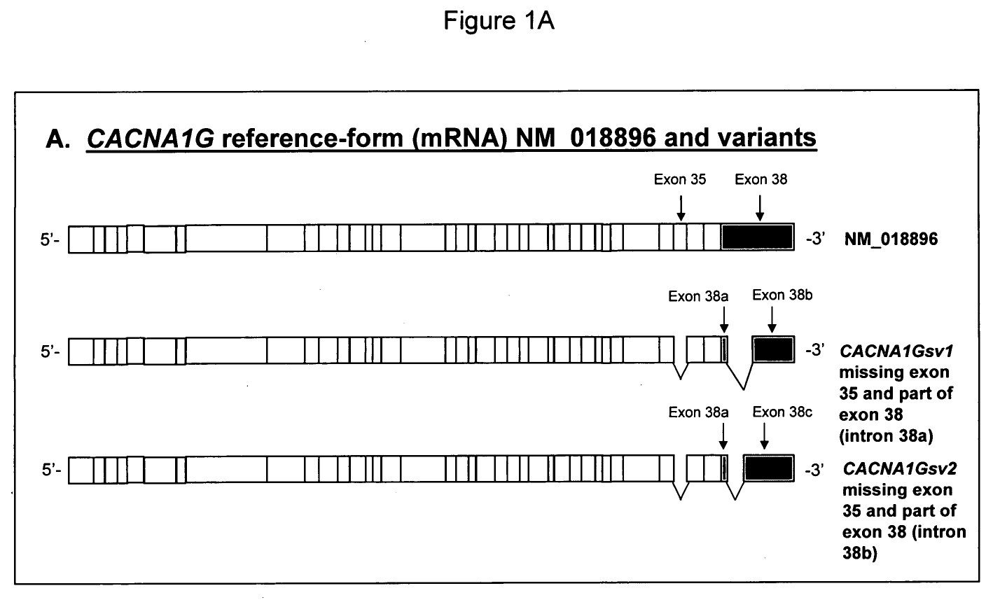

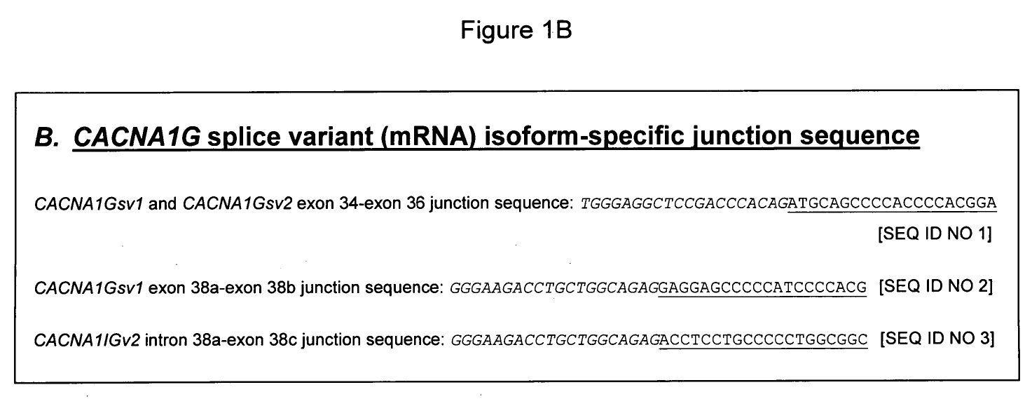

In embodiments where, for example, CACNA1Gsv1 or CACNA1Gsv2 polynucleotide probes are used in hybridization assays to specifically detect the presence of CACNA1Gsv1 or CACNA1Gsv2 polynucleotides in samples, the CACNA1Gsv1 or CACNA1Gsv2 polynucleotides comprise at least 20 nucleotides of the CACNA1Gsv1 or CACNA1Gsv2 sequence that correspond to the respective novel exon junction or novel polynucleotide regions. In particular, for detection of CACNA1Gsv1, the probe comprises at least 20 nucleotides of the CACNA1Gsv1 sequence that corresponds to an exon junction polynucleotide created by the alternative splicing of exon 38a to exon 38b of the primary transcript of the CACNA1G gene (see FIGS. 1A and 1B). For example, the polynucleotide sequence: 5′ GCTGGCAGAGGAGGAGCCCC 3′ [SEQ ID NO 11] represents one embodiment of such an inventive CACNA1Gsv1 polynucleotide wherein a first 10 nucleotide region is complementary and hybridizable to the 3′ end of exon 38a of the CACNA1G gene and a second 10 nucleotide region is complementary and hybridizable to the 5′ end of exon 38b of the CACNA1G gene (see FIG. 1B).

In another embodiment, for the detection of CACNA1Gsv2, the probe comprises at least 20 nucleotides of the CACNA1Gsv2 sequence that corresponds to an exon junction polynucleotide created by the alternative splicing of exon 38a to exon 38c of the primary transcript of the CACNA1G gene (see FIGS. 1A and 1B). For example, the polynucleotide sequence: 5′ GCTGGCAGAGACCTCCTGCC 3′ [SEQ ID NO 12] represents one embodiment of such an inventive CACNA1Gsv2 polynucleotide wherein a first 10 nucleotide region is complementary and hybridizable to the 3′ end of exon 38a of the CACNA1G gene and a second 10 nucleotide region is complementary and hybridizable to the 5′ end of exon 38c of the CACNA1G gene (see FIG. 1B).

In another embodiment, for the detection of both CACNA1Gsv1 and CACNA1Gsv2, the probe comprises at least 20 nucleotides of the CACNA1Gsv1 and CACNA1Gsv2 sequence that corresponds to an exon junction polynucleotide created by the alternative splicing of exon 34 to exon 36 of the primary transcript of the CACNA1G gene (see FIGS. 1A and 1B). For example, the polynucleotide sequence: 5′ CGACCCACAGATGCAGCCCC 3′ [SEQ ID NO 13] represents one embodiment of such an inventive CACNA1Gsv1 and CACNA1Gsv2 polynucleotide wherein a first 10 nucleotide region is complementary and hybridziable to the 3′ end of exon 34 of the CACNA1G gene and a second 10 nucleotide region is complementary and hybridizable to the 5′ end of exon 36 of the CACNA1G gene (see FIG. 1B).

In some embodiments, the first 20 nucleotides of a CACNA1Gsv1 probe comprise a first continuous region of 5 to 15 nucleotides that is complementary and hybridizable to the 5′ end of exon 38a and a second continuous region of 5 to 15 nucleotides that is complementary and hybridizable to the 5′ end of exon 38b of the CACNA1G gene. In some embodiments, the first 20 nucleotides of a CACNA1Gsv2 probe comprise a first continuous region of 5 to 15 nucleotides that is complementary and hybridizable to the 3′ end of exon 38a and a second continuous region of 5 to 15 nucleotides that is complementary and hybridizable to the 5′ end of exon 38c. In some embodiments, the first 20 nucleotides of a CACNA1Gsv1 and CACNA1Gsv2 probe comprise a first continuous region of 5 to 15 nucleotides that is complementary and hybridizable to the 5′ end of exon 34 and a second continuous region of 5 to 15 nucleotides that is complementary and hybridizable to the 5′ end of exon 36 of the CACNA1G gene.

In other embodiments, the CACNA1Gsv1 or CACNA1Gsv2 polynucleotide comprises at least 40, 60, 80 or 100 nucleotides of the CACNA1Gsv1 or CACNA1Gsv2 sequence, respectively, that correspond to a junction polynucleotide region created by the alternative splicing of exon 38a to exon 38b in the case of CACNA1Gsv1; or in the case of CACNA1Gsv2, the alternative splicing of exon 38a to exon 38c; or in the case of both CACNA1Gsv1 and CACNA1Gsv2, the alternative splicing of exon 34 to exon 36 of the primary transcript of the CACNA1G gene. In embodiments involving CACNA1Gsv1, the CACNA1Gsv1 polynucleotide is selected to comprise a first continuous region of at least 5 to 15 nucleotides that is complementary and hybridizable to the 3′ end of exon 38a and a second continuous region of at least 5 to 15 nucleotides that is complementary and hybridizable to the 5′ end of exon 38b of the CACNA1G gene. Similarly, in embodiments involving CACNA1Gsv2, the CACNA1Gsv2 polynucleotide is selected to comprise a first continuous region of at least 5 to 15 nucleotides that is complementary and hybridizable to the 3′ end of exon 38a and a second continuous region of at least 5 to 15 nucleotides that is complementary and hybridizable to the 5′ end of exon 38c of the CACNA1G gene. In embodiments involving CACNA1Gsv1 and CACNA1Gsv2, the CACNA1Gsv1 and CACNA1Gsv2 polynucleotide is selected to comprise a first continuous region of at least 5 to 15 nucleotides that is complementary and hybridizable to the 3′ end of exon 34 and a second continuous region of at least 5 to 15 nucleotides that is complementary and hybridizable to the 5′ end of exon 36 of the CACNA1G gene. As will be apparent to a person of skill in the art, a large number of different polynucleotide sequences from the region of the exon 38a to exon 38b, exon 38a to exon 38c, or exon 34 to exon 36 splice junctions may be selected which will, under appropriate hybridization conditions, have the capacity to detectably hybridize to CACNA1Gsv1 or CACNA1Gsv2 polynucleotides and yet will hybridize to a much less extent or not at all to CACNA1G isoform polynucleotides wherein exon 38a is not spliced to exon 38b, wherein exon 38a is not spliced to exon 38c, or wherein exon 34 is not spliced to exon 36.

Preferably, non-complementary nucleic acid that is present has a particular purpose such as being a reporter sequence or being a capture sequence. However, additional nucleic acid need not have a particular purpose as long as the additional nucleic acid does not prevent the CACNA1Gsv1 or CACNA1Gsv2 nucleic acid from distinguishing between target polynucleotides, e.g., CACNA1Gsv1 or CACNA1Gsv2 polynucleotides, and non-target polynucleotides, including, but not limited to CACNA1G polynucleotides not comprising the exon 34 to exon 36 splice junction found in CACNA1Gsv1 and CACNA1Gsv2, or the exon 38a to exon 38b or exon 38a to exon 38c splice junctions found in CACNA1Gsv1 or CACNA1Gsv2, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

CACNA1Gsv1 or CACNA1Gsv2 polynucleotides, such as those comprising SEQ ID NO 4 or SEQ ID NO 6, respectively, can be used to make CACNA1Gsv1 or CACNA1Gsv2 polypeptides, respectively. In particular, CACNA1Gsv1 or CACNA1Gsv2 polypeptides can be expressed from recombinant nucleic acids in a suitable host or in vitro using a translation system. Recombinantly expressed CACNA1Gsv1 or CACNA1Gsv2 polypeptides can be used, for example, in assays to screen for compounds that bind CACNA1Gsv1 or CACNA1Gsv2, respectively. Alternatively, CACNA1Gsv1 or CACNA1Gsv2 polypeptides can also be used to screen for compounds that bind to one or more CACNA1G or calcium channel isoforms, but do not bind to CACNA1Gsv1 or CACNA1Gsv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pRS416 (ATCC 87521), pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad, Calif.).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

Recombinant DNA molecules that feature precise fusions of polynucleotide sequences can also be assembled using standard recombinational subcloning techniques. Recombination-mediated, PCR-directed, or PCR-independent plasmid construction in yeast is well known in the art (see Hua et al., 1997, Plasmid 38:91-96; Hudson et al., 1997, Genome Res. 7(12):1169-1173; Oldenburg et al., 1997, Nucleic Acids Res. 25(2):451-452; Raymond et al., 1999, BioTechniques 26(1)134-8, 140-1). Overlapping sequences between the donor DNA fragments and the acceptor plasmid permit recombination in yeast. An example of recombination-mediated plasmid construction in Saccharomyces cerevisiae is described in Oldenburg et al., 1997, Nucleic Acids Res. 25(2):451-452: a DNA segment of interest was amplified by PCR so that the PCR product had 20-40 bp of homology at each end to the region of the plasmid at which recombination was to occur. The PCR product and linearized plasmid were co-transformed into yeast, and recombination resulted in replacement of the region between the homologous sequences on the plasmid with the region carried by the PCR fragment. The recombinational method of plasmid construction bypasses the need for extensive modification and ligation steps and does not rely on available restriction sites. These cloning vectors can then be utilized for protein expression in multiple systems.

To enhance expression in a particular host it may be useful to modify the sequence provided in SEQ ID NO 4 or SEQ ID NO 6 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

CACNA1Gsv1 and CACNA1Gsv2 Polypeptides

CACNA1Gsv1 polypeptides contain an amino acid sequence comprising, consisting or consisting essentially of SEQ ID NO 5. CACNA1Gsv2 polypeptides contain an amino acid sequence comprising, consisting or consisting essentially of SEQ ID NO 7. CACNA1Gsv1 [SEQ ID NO 5] has 95% amino acid sequence identity as compared to the CACNA1G reference protein (P_{—}061496). CACNA1Gsv2 [SEQ ID NO 7] has 91% amino acid sequence identity as compared to the CACNA1G reference protein (NP_{—}061496). CACNA1Gsv1 or CACNA1Gsv2 polypeptides have a variety of uses, such as providing a marker for the presence of CACNA1Gsv1 or CACNA1Gsv2, respectively; use as an immunogen to produce antibodies binding to CACNA1Gsv1 or CACNA1Gsv2, respectively; use as a target to identify compounds binding selectively to CACNA1Gsv1 or CACNA1Gsv2, respectively; or use in an assay to identify compounds that bind to one or more CACNA1G or calcium channel isoforms but do not bind to or interact with CACNA1Gsv1 or CACNA1Gsv2, respectively.

In chimeric polypeptides containing one or more regions from CACNA1Gsv1 or CACNA1Gsv2 and one or more regions not from CACNA1Gsv1 or CACNA1Gsv2, respectively, the region(s) not from CACNA1Gsv1 or CACNA1Gsv2 can be used, for example, to achieve a particular purpose or to produce a polypeptide that can substitute for CACNA1Gsv1, or CACNA1Gsv2 or fragments thereof. Particular purposes that can be achieved using chimeric CACNA1Gsv1 or CACNA1Gsv2 polypeptides include providing a marker for CACNA1Gsv1 or CACNA1Gsv2 activity, respectively, and altering the activity and regulation of the CACNA1G calcium channel.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional CACNA1Gsv1 and CACNA1Gsv2

Functional CACNA1Gsv1 and CACNA1Gsv2 are different protein isoforms of CACNA1G. The identification of the amino acid and nucleic acid sequences of CACNA1Gsv1 or CACNA1Gsv2 provides tools for obtaining functional proteins related to CACNA1Gsv1 or CACNA1Gsv2, respectively, from other sources, for producing CACNA1Gsv1 or CACNA1Gsv2 chimeric proteins, and for producing functional derivatives of SEQ ID NO 5 or SEQ ID NO 7.

CACNA1Gsv1 or CACNA1Gsv2 polypeptides can be readily identified and obtained based on their sequence similarity to CACNA1Gsv1 [SEQ ID NO 5] or CACNA1Gsv2 [SEQ ID NO 7], respectively. In particular, CACNA1Gsv1 polypeptide lacks amino acids, encoded by exon 35 and encoded by a portion of exon 38, referred to as intron 38a, of the CACNA1G gene. The deletion of exon 35 and the splicing of exon 34 to exon 36, and the deletion of intron 38a and the splicing of exon 38a to exon 38b of the CACNA1G heteronuclear RNA (hnRNA) transcript do not alter the protein reading frame at the exon 34 to exon 36 and exon 38a to exon 38b splice junctions. Thus, the CACNA1Gsv1 polypeptide lacks 45 amino acids encoded by nucleotides corresponding to exon 35 and lacks 79 amino acids encoded by nucleotides corresponding to intron 38a of the CACNA1G hnRNA as compared to the CACNA1G reference protein (NP_{—}061496). The CACNA1Gsv2 polypeptides lack amino acids, encoded by exon 35 and encoded by a portion of exon 38, referred to as intron 38b, of the CACNA1G gene. The deletion of exon 35 and the splicing of exon 34 to exon 36 of the CACNA1G hnRNA transcript do not alter the protein reading frame at the exon 34 to exon 36 splice junction. The deletion of intron 38b and the splicing of exon 38a to exon 38c of the CACNA1G hnRNA do alter the protein reading frame at the exon 38a to exon 38c splice junction, introducing a stop codon 49 nucleotides following the exon 38a-exon 38c splice junction. Thus, the CACNA1Gsv2 polypeptide lacks 45 amino acids encoded by nucleotides corresponding to exon 35, lacks 71 amino acids encoded by nucleotides corresponding to intron 38b of the CACNA1G hnRNA, and contains 16 unique amino acids encoded by the nucleotides located after the splice junction which results from the splicing of exon 38a to exon 38c as compared to the CACNA1G reference protein (NP_{—}061496).

Both the amino acid and nucleic acid sequences of CACNA1Gsv1 or CACNA1Gsv2 can be used to help identify and obtain CACNA1Gsv1 or CACNA1Gsv2 polypeptides, respectively. For example, SEQ ID NO 4 can be used to produce degenerative nucleic acid probes or primers for identifying and cloning nucleic acid polynucleotides encoding for a CACNA1Gsv1 polypeptide. In addition, polynucleotides comprising, consisting, or consisting essentially of SEQ ID NO 4 or fragments thereof, can be used under conditions of moderate stringency to identify and clone nucleic acids encoding CACNA1Gsv1 polypeptides from a variety of different organisms. The same methods can also be performed with polynucleotides comprising, consisting, or consisting essentially of SEQ ID NO 6, or fragments thereof, to identify and clone nucleic acids encoding CACNA1Gsv2.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with CACNA1Gsv1 or CACNA1Gsv2 obtained from a particular source, derivatives can be produced. Such derivatives include polypeptides with amino acid substitutions, additions and deletions. Changes to CACNA1Gsv1 or CACNA1Gsv2 to produce a derivative having essentially the same properties should be made in a manner not altering the tertiary structure of CACNA1Gsv1 or CACNA1Gsv2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

CACNA1Gsv1 and CACNA1Gsv2 Antibodies

Antibodies recognizing CACNA1Gsv1 or CACNA1Gsv2 can be produced using a polypeptide containing SEQ ID NO 5 in the case of CACNA1Gsv1 or SEQ ID NO 7 in the case of CACNA1Gsv2, respectively, or a fragment thereof as an immunogen. Preferably, a CACNA1Gsv1 polypeptide used as an immunogen consists of a polypeptide of SEQ ID NO 5 or a SEQ ID NO 5 fragment having at least 10 contiguous amino acids in length corresponding to the polynucleotide region representing the junction from exon 38a to exon 38b of the CACNA1G gene. When CACNA1Gsv2 polypeptide is used as an immunogen, preferably it consists of a polypeptide derived from SEQ ID NO 7 or a SEQ ID NO 7 fragment, having at least 10 contiguous amino acids in length corresponding to the polynucleotide region representing the junction from exon 38a to exon 38c of the CACNA1G gene.

In some embodiments where, for example, CACNA1Gsv1 polypeptides are used to develop antibodies that bind specifically to CACNA1Gsv1 and not to other isoforms of CACNA1G, the CACNA1Gsv1 polypeptides comprise at least 10 amino acids of the CACNA1Gsv1 polypeptide sequence corresponding to a junction polynucleotide region created by the alternative splicing of exon 38a to exon 38b of the primary transcript of the CACNA1G gene (see FIG. 1). For example, the amino acid sequence: amino terminus-DLLAEEEPPS-carboxy terminus [SEQ ID NO 14] represents one embodiment of such an inventive CACNA1Gsv1 polypeptide wherein a first 5 amino acid region is encoded by a nucleotide sequence at the 3′ end of exon 38a of the CACNA1G gene and a second 5 amino acid region is encoded by the nucleotide sequence at the 5′ end of exon 38b. Preferably, at least 10 amino acids of the CACNA1Gsv1 polypeptide comprise a first continuous region of 2 to 8 amino acids that is encoded by nucleotides at the 3′ end of exon 38a and a second continuous region of 2 to 8 amino acids that is encoded by nucleotides at the 5′ end of exon 38b.

In other embodiments where, for example, CACNA1Gsv2 polypeptides are used to develop antibodies that bind specifically to CACNA1Gsv2 and not to other CACNA1G isoforms, the CACNA1Gsv2 polypeptides comprise at least 10 amino acids of the CACNA11Gsv2 polypeptide sequences corresponding to a junction polynucleotide region created by the alternative splicing of exon 38a to exon 38c of the primary transcript of the CACNA1G gene (see FIG. 1). For example, the amino acid sequence: amino terminus-DLLAETSCPL-carboxy terminus [SEQ ID NO 15] represents one embodiment of such an inventive CACNA1Gsv2 polypeptide wherein a first 5 amino acid region is encoded by a nucleotide sequence at the 3′ end of exon 38a of the CACNA1G gene and a second 5 amino acid region is encoded by the nucleotide sequence at the 5′ end of exon 38c. Preferably, at least 10 amino acids of the CACNA1Gsv2 polypeptide comprise a first continuous region of 2 to 8 amino acids that is encoded by nucleotides at the 3′ end of exon 38a and a second continuous region of 2 to 8 amino acids that is encoded by nucleotides at the 5′ end of exon 38c. Alternatively, in the case of CACNA1Gsv1 [SEQ ID NO 5] and CACNA1Gsv2 [SEQ ID NO 7], the amino acid sequence: amino terminus-GSDPQMQPHP-carboxy terminus [SEQ ID NO 16], represent one embodiment of such an inventive CACNA1Gsv1 and CACNA1Gsv2 polypeptide wherein a first 5 amino acid region is coded by a nucleotide sequence at the 3′ end of exon 34 of the CACNA1G gene and a second 5 amino acid region is coded by a nucleotide sequence at the 5′ end of exon 36. Preferably, at least 10 amino acids of the CACNA1Gsv1 and CACNA1Gsv2 polypeptide comprises a first continuous region of 2 to 8 amino acids that is coded by nucleotides at the 3′ end of exon 34 and a second continuous region of 2 to 8 amino acids that is coded by nucleotides at the 5′ end of exon 36.

In other embodiments, CACNA1Gsv1-specific antibodies are made using a CACNA1Gsv1 polypeptide that comprises at least 20, 30, 40 or 50 amino acids of the CACNA1Gsv1 sequence that corresponds to a junction polynucleotide region created by the alternative splicing of exon 38a to exon 38b of the primary transcript of the CACNA1G gene. In each case the CACNA1Gsv1 polypeptides are selected to comprise a first continuous region of at least 5 to 15 amino acids that is encoded by nucleotides at the 3′ end of exon 38a and a second continuous region of 5 to 15 amino acids that is encoded by nucleotides directly after the novel splice junction created by the splicing of exon 38a to exon 38b of the CACNA1G gene.

In other embodiments, CACNA1Gsv2-specific antibodies are made using a CACNA1Gsv2 polypeptide that comprises at least 20, 30, 40 or 50 amino acids of the CACNA1Gsv2 sequence that corresponds to a junction polynucleotide region created by the alternative splicing of exon 38a to exon 38c of the primary transcript of the CACNA1G gene. In each case the CACNA1Gsv2 polypeptides are selected to comprise a first continuous region of at least 5 to 15 amino acids that is encoded by nucleotides at the 3′ end of exon 38a and a second continuous region of 5 to 15 amino acids that is encoded by nucleotides directly after the novel splice junction created by the splicing of exon 38a to exon 38c of the CACNA1G gene.

In other embodiments, CACNA1Gsv1 and CACNA1Gsv2-specific antibodies are made using a CACNA1Gsv1 and CACNA1Gsv2 polypeptide that comprises at least 20, 30, 40 or 50 amino acids of the CACNA1Gsv1 and CACNA1Gsv2 sequence that corresponds to a junction polynucleotide region created by the alternative splicing of exon 34 to exon 36 of the primary transcript of the CACNA1G gene. In each case the CACNA1Gsv1 and CACNA1Gsv2 polypeptides are selected to comprise a first continuous region of at least 5 to 15 amino acids that is encoded by nucleotides at the 3′ end of exon 34 and a second continuous region of 5 to 15 amino acids that is encoded by nucleotides directly after the novel splice junction created by the splicing of exon 34 to exon 36 of the CACNA1G gene.

Antibodies to CACNA1Gsv1 or CACNA1Gsv2 have different uses, such as to identify the presence of CACNA1Gsv1 or CACNA1Gsv2, respectively, and to isolate CACNA1Gsv1 or CACNA1Gsv2 polypeptides, respectively. Identifying the presence of CACNA1Gsv1 can be used, for example, to identify cells producing CACNA1Gsv1. Such identification provides an additional source of CACNA1Gsv1 and can be used to distinguish cells known to produce CACNA1Gsv1 from cells that do not produce CACNA1Gsv1. For example, antibodies to CACNA1Gsv1 can distinguish human cells expressing CACNA1Gsv1 from human cells not expressing CACNA1Gsv1 or non-human cells (including bacteria) that do not express CACNA1Gsv1. Such CACNA1Gsv1 antibodies can also be used to determine the effectiveness of CACNA1Gsv1 ligands, using techniques well known in the art, to detect and quantify changes in the protein levels of CACNA1Gsv1 in cellular extracts, and in situ immunostaining of cells and tissues. In addition, the same above-described utilities also exist for CACNA1Gsv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

CACNA1Gsv1 and CACNA1Gsv2 Binding Assay

A number of compounds known to modulate calcium channel activity have been disclosed, including mibefradil, kurotoxin, succinimide, and nickel (Martin et al., 2000, J. Pharmacol. Exp. Ther. 295:302-308; Chuang et al., 1998, Nature Neurosci. 1:668-674; Gomora et al., 2001, Mol Pharmacol. 60:1121-1132; Lee et al., 1999, Biophys. J. 77:3034-3042). Methods for expressing calcium channels in Xenopus oocytes or HEK293 cells and monitoring the activity of these channels, including analyzing the effect of compounds on the activity of calcium channel activity, have been described previously (Zhuang et al., 2000, Diabetes 49:59-64; Perez-Reyes et al., 1998, Nature 391:896-900; WO 99/29847; Lee et al., 1999, Biophys. J. 77:3034-3042; Lee et al., 1999, J. Neurosci. 19:1912-1921; Gomora et al., 2002, Biophys. J. 83: 229-241; Martin et al., 2000, 295:302-308; McRory et al., 276:3999-4011). US2004/0175761 describes compositions and methods of use for the voltage sensor domain of ion channel proteins, immobilized on a solid support, for screening compounds that bind to voltage-dependent ion channels. Methods for screening compounds for their effects on ion channel activity have also been disclosed (see for example US 2002/0025568, US 2002/0045159, WO 03/006103). A person skilled in the art may use these methods, and others, to screen CACNA1Gsv1 or CACNA1Gsv2 polypeptides for compounds that bind to, and in some cases functionally alter, each CACNA1G isoform protein.

CACNA1Gsv1, CACNA1Gsv2, or fragments thereof, can be used in binding studies to identify compounds binding to or interacting with CACNA1Gsv1, CACNA1GSv2, or fragments thereof, respectively. In one embodiment, CACNA1Gsv1, or a fragment thereof, can be used in binding studies with a calcium channel isoform protein, or a fragment thereof, to identify compounds that: bind to or interact with CACNA1Gsv1 and other calcium channel isoforms; bind to or interact with one or more other calcium channel isoforms and not with CACNA1Gsv1; bind to or interact with CACNA1Gsv1 and not with one or more other calcium channel isoforms. A similar series of compound screens can, of course, also be performed using CACNA1Gsv2 rather than, or in addition to, CACNA1Gsv1. Such binding studies can be performed using different formats including competitive and non-competitive formats. Further competition studies can be carried out using additional compounds determined to bind to CACNA1Gsv1, CACNA1Gsv2, other CACNA1G isoforms, or other calcium channel isoforms.

The particular CACNA1Gsv1 or CACNA1Gsv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using CACNA1Gsv1 expressed from a recombinant nucleic acid. Alternatively, recombinantly expressed CACNA1Gsv1 consists of the SEQ ID NO 5 amino acid sequence. In addition, binding studies are performed using CACNA1Gsv2 expressed from a recombinant nucleic acid. Alternatively, recombinantly expressed CACNA1Gsv2 consists of the SEQ ID NO 7 amino acid sequence.

Binding assays can be performed using individual compounds or preparations containing different numbers of compounds. A preparation containing different numbers of compounds having the ability to bind to CACNA1Gsv1 or CACNA1Gsv2 can be divided into smaller groups of compounds that can be tested to identify the compound(s) binding to CACNA1Gsv1 or CACNA1Gsv2, respectively.

Binding assays can be performed using recombinantly produced CACNA1Gsv1 or CACNA1Gsv2 present in different environments. Such environments include, for example, cell extracts and purified cell extracts containing a CACNA1Gsv1 or CACNA1Gsv2 recombinant nucleic acid; and also include, for example, the use of a purified CACNA1Gsv1 or CACNA1Gsv2 polypeptide produced by recombinant means which is introduced into different environments.

In one embodiment of the invention, a binding method is provided for screening for a compound able to bind selectively to CACNA1Gsv1. The method comprises the steps: providing a CACNA1Gsv1 polypeptide comprising SEQ ID NO 5; providing a calcium channel isoform polypeptide that is not CACNA1Gsv1; contacting the CACNA1Gsv1 polypeptide and the calcium channel isoform polypeptide that is not CACNA1Gsv1 with a test preparation comprising one or more test compounds; and then determining the binding of the test preparation to the CACNA1Gsv1 polypeptide and to the calcium channel isoform polypeptide that is not CACNA1Gsv1, wherein a test preparation that binds to the CACNA1Gsv1 polypeptide, but does not bind to the calcium channel isoform polypeptide that is not CACNA1Gsv1, contains one or more compounds that selectively bind to CACNA1Gsv1.

In one embodiment of the invention, a binding method is provided for screening for a compound able to bind selectively to CACNA1Gsv2. The method comprises the steps: providing a CACNA1Gsv2 polypeptide comprising SEQ ID NO 7; providing a calcium channel isoform polypeptide that is not CACNA1Gsv2; contacting the CACNA1Gsv2 polypeptide and the calcium channel isoform polypeptide that is not CACNA1Gsv2 with a test preparation comprising one or more test compounds; and then determining the binding of the test preparation to the CACNA1Gsv2 polypeptide and to the calcium channel isoform polypeptide that is not CACNA1Gsv2, wherein a test preparation that binds to the CACNA1Gsv2 polypeptide, but does not bind to the calcium channel isoform polypeptide that is not CACNA1Gsv2, contains one or more compounds that selectively bind to CACNA1Gsv2.

In another embodiment of the invention, a binding method is provided for screening for a compound able to bind selectively to a calcium channel isoform polypeptide that is not CACNA1Gsv1. The method comprises the steps: providing a CACNA1Gsv1 polypeptide comprising SEQ ID NO 5; providing a calcium channel isoform polypeptide that is not CACNA1Gsv1; contacting the CACNA1Gsv1 polypeptide and the calcium channel isoform polypeptide that is not CACNA1Gsv1 with a test preparation comprising one or more test compounds; and then determining the binding of the test preparation to the CACNA1Gsv1 polypeptide and the calcium channel isoform polypeptide that is not CACNA1Gsv1, wherein a test preparation that binds the calcium channel isoform polypeptide that is not CACNA1Gsv1, but does not bind CACNA1Gsv1, contains a compound that selectively binds the calcium channel isoform polypeptide that is not CACNA1Gsv1. Alternatively, the above method can be used to identify compounds that bind selectively to a calcium channel isoform polypeptide that is not CACNA1Gsv2 by performing the method with CACNA1Gsv2 protein comprising SEQ ID NO 7.

The above-described selective binding assays can also be performed with a polypeptide fragment of CACNA1Gsv1 or CACNA1Gsv2, wherein the polypeptide fragment comprises at least 10 consecutive amino acids that are coded by a nucleotide sequence that bridges the junction created by the splicing of the 3′ end of exon 34 to the 5′ end of exon 36 in the case of CACNA1Gsv1 and CACNA1Gsv2, by the splicing of the 3′ end of exon 38a to the 5′ end of exon 38b in the case of CACNA1Gsv1, or by the splicing of the 3′ end of exon 38a to the 5′ end of exon 38c in the case of CACNA1Gsv2. Similarly, the selective binding assays may also be performed using a polypeptide fragment of a calcium channel isoform polypeptide that is not CACNA1Gsv1 or CACNA1Gsv2, wherein the polypeptide fragment comprises at least 10 consecutive amino acids that are coded by: a) a nucleotide sequence that is contained within exon 35 of the CACNA1G gene; b) a nucleotide sequence that is contained within intron 38a of the CACNA1G gene; or c) a nucleotide sequence that is contained within intron 38b of the CACNA1G gene.

CACNA1G Functional Assays

CACNA1G encodes the alpha subunit of a highly conserved voltage gated calcium channel that is implicated in epilepsy and sleeping disorders. Splice variants of calcium channels may exhibit different current kinetics and different binding affinities for compounds, peptides and other small molecules. The identification of CACNA1Gsv1 and CACNA1Gsv2 as splice variants of CACNA1G provides a means of screening for compounds that bind to CACNA1Gsv1 and/or CACNA1Gsv2 protein thereby altering the activity or regulation of CACNA1Gsv1 and/or CACNA1Gsv2 calcium channels. Assays involving a functional CACNA1Gsv1 or CACNA1Gsv2 polypeptide can be employed for different purposes, such as selecting for compounds active at CACNA1Gsv1 or CACNA1Gsv2; evaluating the ability of a compound to affect the ion channel activity of each respective splice variant; and mapping the activity of different CACNA1Gsv1 and CACNA1Gsv2 regions. CACNA1Gsv1 and CACNA1Gsv2 activity can be measured using different techniques such as: detecting a change in the intracellular conformation of CACNA1Gsv1 or CACNA1Gsv2; detecting a change in the intracellular location of CACNA1Gsv1 or CACNA1Gsv2; or measuring the ion channel activity of CACNA1Gsv1 or CACNA1Gsv2.

Recombinantly expressed CACNA1Gsv1 and CACNA1Gsv2 can be used to facilitate the determination of whether a compound's activity in a cell is dependent upon the presence of CACNA1Gsv1 and CACNA1GSv2. For example, CACNA1Gsv1 or CACNA1Gsv2 can be expressed by an expression vector in a cell line and used in a co-culture growth assay, such as described in U.S. Pat. No. 6,518,035, to identify compounds that alter the growth of the cell expressing CACNA1Gsv1 or CACNA1Gsv2, respectively, from the expression vector as compared to the same cell line but lacking the CACNA1Gsv1 or CACNA1Gsv2 expression vector, respectively. Alternatively, determination of whether a compound's activity on a cell is dependent upon the presence of CACNA1Gsv1 or CACNA1Gsv2 can also be done using gene expression profile analysis methods as described, for example, in U.S. Pat. No. 6,324,479.

Techniques for measuring voltage gated ion channel activity are well known in the art. Methods for expressing calcium channels in Xenopus oocytes and human cells, and monitoring the activity of these channels, including analyzing the effect of compounds on the activity of calcium channel activity, have been described previously (WO 99/29847; U.S. Pat. No. 6,589,787; Lee et al., 1999, Biophys. J. 77:3034-3042; Lee et al., 1999, J. Neurosci. 19:1912-1921; Gomora et al., 2002, Biophys. J. 83: 229-241; Martin et al., 2000, 295:302-308; McRory et al., 276:3999-4011). The patch clamp technique measures ion current through ion channel proteins and can be used to analyze the effect of drugs on ion channel function. The activity of these channels can be measured electrically (single channel recording) or, alternatively, the patch can be ruptured allowing measurements of the combined channel activity of the entire cell membrane (whole cell recording) (Neher et al., 1978, Pflugers Arch. 375: 219-28; Hamill et al., 1981, Pflugers Arch. 391:85-100; Sakman et al., 1984, Annu Rev Physiol. 46: 455-72; Neher et al., 1992, Sci. Am. 266: 44-51). Other methods for measuring ion channel activity include optical reading of voltage-sensitive dyes (Cohen et al., 1978, Annual Reviews of Neuroscience 1:171-82) and extracellular recording of fast events using metal (Thomas et al., 1972, Exp. Cell Res. 74:61-66) or field effect transistor (Fromherz et al., 1991, Science 252:1290-1293) electrodes. High throughput methods for assaying ion channel activity have also been described (see WO 03/006103 and US 2002/0028480). A variety of other assays has been used to investigate the properties of calcium channels and therefore would also be applicable to the measurement of CACNA1Gsv1 or CACNA1Gsv2 function.

CACNA1Gsv1 or CACNA1Gsv2 functional assays can be performed using cells expressing CACNA1Gsv1 or CACNA1Gsv2 at a high level. These proteins will be contacted with individual compounds or preparations containing different compounds. A preparation containing different compounds where one or more compounds affect CACNA1Gsv1 or CACNA1Gsv2 in cells over-producing CACNA1Gsv1 or CACNA1Gsv2 as compared to control cells containing an expression vector lacking CACNA1Gsv1 or CACNA1Gsv2 coding sequences, can be divided into smaller groups of compounds to identify the compound(s) affecting CACNA1Gsv1 or CACNA1Gsv2 activity, respectively.

CACNA1Gsv1 or CACNA1Gsv2 functional assays can be performed using recombinantly produced CACNA1Gsv1 or CACNA1Gsv2 present in different environments. Such environments include, for example, cell extracts and purified cell extracts containing the CACNA1Gsv1 or CACNA1Gsv2 expressed from recombinant nucleic acid; and the use of purified CACNA1Gsv1 or CACNA1Gsv2 produced by recombinant means that is introduced into a different environment suitable for measuring ion channel activity.

Modulating CACNA1Gsv1 and CACNA1GSv2 Expression

CACNA1Gsv1 or CACNA1Gsv2 expression can be modulated as a means for increasing or decreasing CACNA1Gsv1 or CACNA1Gsv2 activity, respectively. Such modulation includes inhibiting the activity of nucleic acids encoding the CACNA1G isoform target to reduce CACNA1G isoform protein or polypeptide expression, or supplying CACNA1G nucleic acids to increase the level of expression of the CACNA1G target polypeptide thereby increasing CACNA1G activity.

Inhibition of CACNA1Gsv1 and CACNA1Gsv2 Activity

CACNA1Gsv1 or CACNA1Gsv2 nucleic acid activity can be inhibited using nucleic acids recognizing CACNA1Gsv1 or CACNA1Gsv2 nucleic acid and affecting the ability of such nucleic acid to be transcribed or translated. Inhibition of CACNA1Gsv1 or CACNA1Gsv2 nucleic acid activity can be used, for example, in target validation studies.

A preferred target for inhibiting CACNA1Gsv1 or CACNA1GSv2 is mRNA stability and translation. The ability of CACNA1Gsv1 or CACNA1Gsv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding region of a gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Methods for using RNAi to modify calcium channel activity have been described previously (Keller et al., 2000, J. Pharmacol. Exp. Ther. 295(1): 367-72). Modified and unmodified nucleic acids can be used as anti-sense molecules, RNAi and ribozymes. Different types of modifications can affect certain-RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing CACNA1Gsv1 and CACNA1Gsv2 Expression

Nucleic acids encoding for CACNA1Gsv1 or CACNA1Gsv2 can be used, for example, to cause an increase in CACNA1G activity or to create a test system (e.g., a transgenic animal) for screening for compounds affecting CACNA1Gsv1 or CACNA1Gsv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of CACNA1Gsv1 and CACNA1Gsv2 Using RT-PCR and Restriction Digest

Deletions of exons 34 and 35 and a partial drop of exon 38 have been reported (Mittman et al., 1999, Neurosci. Lett. 274:143-146). However, the combination of the partial exon 38 drop with the inclusion of exon 34 or 35, or both, have not been reported. Alterations to the C-terminus of the calcium channel may affect current kinetics, but would likely not great affect the channel's function (Chemin et al., 2001, Eur. J. Neurosci. 14:1678-1686; Murbartian et al., 2002, FEBS Lett. 528:272-278; Gomora et al., 2002, Biophys. J. 83:229-241). The structure of CACNA1G mRNA in the region corresponding to exons 33 to 38, which corresponds to S6 of Domain IV and the C-terminus of CACNA1G, was determined for human brain thalamus using an RT-PCR based assay. mRNA isolated from human brain thalamus was obtained from BD Biosciences Clontech (Palo Alto, Calif.) and reverse transcribed using the Superscript II reverse transcription kit (Invitrogen, Carlsbad, Calif.).

Reverse Transcription

2 μg of mRNA from brain thalamus was subjected to a one-step reverse transcription protocol using the Superscript II reverse transcription kit (Invitrogen, Carlsbad, Calif.). The following reaction components were combined in a PCR tube according to manufacturer's instructions:

2 μl dNTPs

18 μl H₂O(RNAse and DNAse free)

2 μl random primer

2 μl brain thalamus mRNA (1 μg/μl)

The reaction mixture was placed in an ABI 2700 Thermal Cycler (Applied Biosystems, Foster City, Calif.) and incubated at 65° C. for 5 minutes. The reaction mixture was then chilled on ice. The following reaction components were then added to the tube:

8 μl Superscript II 5× buffer

4 μl DT

2 μl RNAse OUT

The reaction mixture was placed back in the ABI 2700 Thermal Cycler and incubated at 25° C. for 10 minutes, followed by a 42° C. incubation for 2 minutes. 2 III of Superscript II reverse transcriptase was added to the reaction tube sitting in the ABI 2700 Thermal Cycler, which was followed by a 50 minute incubation at 42° C. and then a 15 minute incubation at 70° C. The reaction tube was chilled on ice. 2 μl of RNAse H (Invitrogen, Carlsbad, Calif.) was added to the reaction mixture, which was then incubated at 37° C. for 20 minutes. The completed reaction mixture containing cDNA was immediately stored at −80° C. until it was used for PCR.

PCR

PCR primers were selected that were complementary in sequences in exon 33 and exon 38 or the reference coding sequences in CACNA1G (NM_{—}018896). Based upon the nucleotide sequence of CACNA1G mRNA, the CACNA1G exon 33 and exon 38 primer set (hereafter CACNA1G₃₃₋₃₈ primer set) was expected to amplify a 1,005 base pair amplicon representing the “reference” CACNA1G mRNA region. The CACNA1G exon 33 forward primer has the sequence: 5′ GATCAGCCTCCCACTTTTCC 3′ [SEQ ID NO 17]; and the CACNA1G exon 38 reverse primer has the sequence: 5′ CCTCCACGCTGTAGCACTTC 3′ [SEQ ID NO 18].

Brain thalamus cDNA from the previously described reverse transcription reaction was subjected to a PCR amplification protocol as follows:
44 μl Platinum PCR Supermix (Invitrogen, Carlsbad, Calif.)
2.5 μl 20 μM CACNA1G exon 33 forward primer
2.5 μl 20 μM CACNA1G exon 38 reverse primer
1 μl brain thalamus cDNA (from previous reverse transcription reaction)

The reaction mixture was placed in an ABI 2700 Thermal Cycler, using the following conditions:
94° C. for 5 minutes;
35 cycles of:
   94° C. for 30 seconds;
   60° C. for 30 seconds;
   72° C. for 30 seconds; then
72° C. for 7 minutes

RT-PCR amplification products (amplicons) were purified with a Qiaquick PCR Purification Kit (Qiagen, Valencia, Calif.) following manufacturer's instructions.

Restriction Digest

In order to identify transcripts which possess a partial drop of exon 38 of the CACNA1G reference sequence (NM_{—}018896), a restriction digest-based selection approach was used. A previously identified partial drop of exon 38 was found to contain a SmaI restriction site. The remaining sequence of the predicted 1,005 base pair amplicon resulting from PCR of the thalamus cDNA using the CACNA1G₃₃₋₃₈ does not contain a SmaI restriction site. SmaI digestion of the amplicon would only cut only those sequences with a full-length exon 38. Any SmaI digested products could not be subsequently cloned using the TOPO® XL PCR Cloning Kit (Invitrogen, Carlsbad, Calif.), as they lack the 3′-A overhangs at the restriction site. Thus, SmaI restriction digest of the RT-PCR amplicon and subsequent cloning into pCR®-XL-TOPO® vector would enrich the yield of clones with inserts which contain a partial drop of exon 38.

The SmaI restriction digest of the RT-PCR amplicion was set up as follows:
10 μl purified RT-PCR amplicon
10 μl SmaI restriction enzyme (Invitrogen, Carlsbad, Calif.)
10 μl React 4 buffer, 10× (Invitrogen, Carlsbad, Calif.)
70 μl H₂O (DNAse and RNAse free)

The restriction digest was placed in an ABI 2700 Thermal Cycler and incubated overnight at 25° C. The restriction digest was inactivated for 20 minutes at 65° C. The restriction digest was then purified with a QIAquick purification kit (Qiagen, Valencia, Calif.) according to manufacturer's instructions. Purified SmaI digest fragments and un-digested exons 33-38 amplicons were cloned into pCR®-XL-TOPO® vector using the reagents and instructions provided with the TOPO® XL PCR Cloning Kit (Invitrogen, Carlsbad, Calif.). Clones were then screened by PCR using the CACNA1G₃₃₋₃₈ primer set and size fractionated on 2% agarose gels to identify any variant insert sizes. Clones were then sequenced from each end (using the same M13 forward and reverse present in the vector sequence) by Lark Technologies (Houston, Tex.).

At least two different RT-PCR amplicons were obtained from human thalamus RNA samples using the CACNA1G₃₃₋₃₈ primer set (data not shown). The thalamus sample assayed exhibited the expected amplicon size of 1005 base pairs for normally spliced CACNA1G mRNA. In addition, the thalamus sample assayed also exhibited an amplicon of about 633 base pairs. Another clone assayed also exhibited an amplicon of about 741 base pairs.

Sequence analysis of the about 633 base pair amplicon revealed that this amplicon form results from the splicing of exon 34 of the CACNA1G hnRNA to exon 36; that is, the coding sequence of exon 35 is completely absent; and from the splicing of exon 38a of the CACNA1G hnRNA to exon 38b; that is, the coding sequence of a portion of exon 38, referred to as intron 38a, is completely absent. This splice variant form was designated CACNA1Gsv1 [SEQ ID NO 4].

Sequence analysis of the about 741 base pair amplicon revealed that this amplicon form results from the splicing of exon 34 of the CACNA1G hnRNA to exon 36; that is, the coding sequence of exon 35 is completely absent; and from the splicing of exon 38a of the CACNA1G hnRNA to exon 38c; that is, the coding sequence of a unique portion of exon 38, referred to as intron 38b, is completely absent. This splice variant form was designated CACNA1Gsv2 [SEQ ID NO 6]. Thus, the RT-PCR results suggested that CACNA1G mRNA in thalamus tissue is composed of a mixed population of molecules wherein at least two of the CACNA1G mRNA splice junctions are altered.

### Example 2

### Cloning of CACNA1Gsv1 and CACNA1Gsv2

RT-PCR and sequencing data indicate that in addition to the normal CACNA1GI reference mRNA sequence, NM_{—}018896, encoding CACNA1G protein, NP_{—}061496, two novel splice variant forms of CACNA1G mRNA also exists in thalamus tissue.

Clones having a nucleotide sequence comprising the splice variants identified in Example 1 (hereafter referred to as CACNA1Gsv1 or CACNA1Gsv2) are isolated using recombination-mediated, PCR-directed plasmid construction in yeast. Recombinant CACNA1Gsv1 or CACNA1Gsv2 DNA molecules featuring precise fusions of polynucleotide sequences may be cloned using recombination-mediated techniques in yeast as previously described in US 2005/0266469, “Alternatively Spliced Isoforms of Checkpoint Kinase 1 (CHK1)” filed May 25, 2005 and US 2005/0227270, “Alternatively Spliced Isoforms of Sodium Channel, Voltage Gated, Type XI, Alpha (SCN11A)” filed Mar. 16, 2005. Recombination-mediated, PCR-directed, or PCR-independent plasmid construction in yeast is well known in the art (see Hua et al., 1997, Plasmid 38:91-96; Hudson et al., 1997, Genome Res. 7:1169-1173; Oldenburg et al., 1997, Nucleic Acids Res. 25:451-452; Raymond et al., 1999, BioTechniques 26:134-8, 140-1).

The polynucleotide sequence of CACNA1Gsv1 mRNA [SEQ ID NO 4] lacks a 135 base pair region corresponding to exon 35 and a 237 base pair region corresponding to intron 38a of the CACNA1G gene. Deletion of the 135 base pair region and 237 base pair region does not alter the protein translation reading frame. Therefore, the CACNA1Gsv1 polypeptide is lacking an internal 45 amino acid region corresponding to exon 35 and an internal 79 amino acid region corresponding to intron 38a of the full length coding sequence of the reference CACNA1G mRNA (NM_{—}018896).

The polynucleotide sequence of CACNA1Gsv2 mRNA [SEQ ID NO 6] lacks a 135 base pair region corresponding to exon 35 and a 214 base pair region corresponding to intron 38b of the CACNA1G gene. Deletion of the 135 base pair region does not alter the protein translation reading frame. However, deletion of the 214 base pair region does alter the protein translation reading frame. Deletion of the 214 base pair region does introduce a stop codon 49 nucleotides after the exon 38a-exon 38c splice junction. Therefore, in addition to lacking 45 amino acids corresponding to exon 35 and 71 amino acids corresponding to intron 38b, the CACNA1Gsv2 polypeptide also possesses a unique 16 amino acid region at the C-terminus compared to the reference CACNA1G mRNA (NM_{—}018896).

### Example 3

### Analyzing Ion Channel Activity of CACNA1G and Other Calcium Channel Isoforms Electrophysiological Analysis of Injected Oocytes

To express CACNA1G and other calcium channel isoform channels in Xenopus oocytes, cDNA encoding the appropriate channel is subcloned into standard expression vectors, such as pGEM-HEA. pGEM-HEA vector was initially created from pGEM-HE and contains 5′ and 3′ untranslated regions from a Xenopus β-globin gene, resulting in high levels of expression (Liman et al., 1992, Neuron 9:861-871). Plasmids are linearized, and capped cRNA is transcribed using the T7 (or SP6) RNA polymerase and the mMessage mMachine kit from Ambion (Austin, Tex.). Xenopus laevis oocytes are prepared using standard techniques (Bernal et al., 1997, J. Pharmacol. Exp. Ther. 282:172-180). Each oocyte is injected with 2-10 ng capped cRNA in a volume of 50 nl and incubated for at least 4 days prior to recording.

Cells are voltage clamped using a two-microelectrode voltage clamp amplifier (OC-725B; Warner Instrument Corp., Hampden, Conn.) as described (Bernal et al., 1997, J. Pharmacol. Exp. Ther. 282:172-180). The standard bath solution contains the following: 10 mM Ba(OH)₂, 90 mM NaOH, 1 mM KOH, and 5 mM HEPES, adjusted to pH 7.4 with methanesulfonic acid. Voltage and current electrodes (0.5-1.5 MΩ tip resistance) contains an agarose cushion and are filled with 3 M KCl (Schreibmayer et al., 1994, Pflugers Arch. 426:453-458). Data are acquired at 5 kHz with the pCLAMP system (Digidata 1200 and pCLAMP 6.0; Axon Instruments, Foster City, Calif.) and filtered at 1 kHz (no. 902 filter, Frequency Devices, Haverhill, Mass.).

Cell Culture and HEK Cell Electrophysiology:

For whole-cell voltage-clamp recordings, stably transfected HEK-293 cells or other eukaryotic cell lines expressing CACNA1G or other calcium channel isoforms are used. Retro-virus vectors may be used to generate cell lines expressing CACNA1G or other calcium channel isoform cell lines. The CACNA1G or other calcium channel isoform is cloned in a retroviral expression vector (pLCNX, Clontech). Subsequently virus particles are used to infect HEK-293 cells, and a cell line stably expressing the CACNA1G channel is selected. Cells are maintained in DMEM (Dulbecco's modified Eagles medium) supplemented with 10% fetal bovine serum (FBS), penicillin (100 U/ml), and streptomycin (100 μg/ml). Cells are plated on poly-D-lysine coated cover slips 4 hours-2 days prior to recording and are then washed with the external solution immediately prior to recording to remove any endogenous redox agents in the FBS.

Single cell recordings of currents through voltage-activated CACNA1G or other calcium channel isoforms are performed at room temperature (20-22° C.) using the whole cell patch-clamp technique (Hamill et al., 1981, Pflugers Archives 391: 85-100). The recording solution contained the following: 10 mM BaCl₂, 140 mM tetraethylammonium (TEA) chloride, 6 mM CsCl, and 10 mM HEPES (pH adjusted to 7.4 with TEA-OH). The standard internal pipette solution contains the following: 55 mM CsCl, 75 mM CsMeSO₄, 10 mM MgCl₂, 0.1 mM EGTA, and 10 mM HEPES (pH adjusted to 7.2 with CsOH). Under these solution conditions, the pipette resistance is typically 1.5-2.0 MΩ. Whole cell currents are recorded from ruptured patches, using an Axopatch 200A amplifier, Digidata 1200 A/D converter, and pCLAMP 6.0 software (Axon Instruments). Data are digitized at 4 kHz and filtered at 1 kHz. Pipettes are made from TW-150-6 capillary tubing (World Precision Instruments Inc., Sarasota, Fla.), using a model P-97 Flaming-Brown pipette puller (Sutter Instrument Co., Novato, Calif.). Series resistance (correction and prediction) and cell capacitance are compensated by at least 80%. Data are analyzed using Clampfit software (Axon Instruments), Excel (Microsoft), and Prism (GraphPad) and are given as mean±standard error mean.

Data are collected using standard pulse protocols and are analyzed to measure calcium current properties that include voltage-dependence, steady-state characteristics, kinetics, and re-priming. Methods of electrophysiological measurements and analysis have been previously described and are known in the art (Lee et al., 1999, Biophys. J. 77:3034-3042; Lee et al., 1999, J. Neurosci. 19:1912-1921; Chemin et al., 2001, EMBO J. 20:7033-7040; Chemin et al., 2001, Biophys. J. 80:1238-1250; Chemin et al., 2001, Eur. J. Neurosci. 14:1678-1686). These measurements are carried out both in control cells expressing CACNA1G or other calcium channel isoforms, and in cells expressing CACNA1G or other calcium channel isoforms that also have been exposed to the compound to be tested.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 4, or the complement thereof.

2. The purified nucleic acid of claim 1, wherein said nucleic acid comprises a sequence encoding SEQ ID NO 5.

3. The purified nucleic acid of claim 1, wherein said nucleic acid encodes a polypeptide consisting of SEQ ID NO 5.

4. A purified polypeptide comprising SEQ ID NO 5.

5. The polypeptide of claim 4, wherein said polypeptide consists of SEQ ID NO 5.

6. An expression vector comprising a nucleotide sequence encoding SEQ ID NO 5, wherein said nucleotide sequence is transcriptionally coupled to an exogenous promoter.

7. The expression vector of claim 6, wherein said nucleotide sequence encodes a polypeptide consisting of SEQ ID NO 5.

8. The expression vector of claim 6, wherein said nucleotide sequence comprises SEQ ID NO 4.

9. The expression vector of claim 6, wherein said nucleotide sequence consists of SEQ ID NO 4.

10. A method of screening for compounds able to bind selectively to CACNA1Gsv1 comprising the steps of:
(a) providing a CACNA1Gsv1 polypeptide comprising SEQ ID NO 5;
(b) providing one or more calcium channel isoform polypeptides that are not CACNA1Gsv1;
(c) contacting said CACNA1Gsv1 polypeptide and said calcium channel isoform polypeptide that is not CACNA1Gsv1 with a test preparation comprising one or more compounds; and
(d) determining the binding of said test preparation to said CACNA1Gsv1 polypeptide and to said calcium channel isoform polypeptide that is not CACNA1Gsv1, wherein a test preparation which binds to said CACNA1Gsv1 polypeptide, but does not bind to said calcium channel isoform polypeptide that is not CACNA1Gsv1, contains a compound that selectively binds said CACNA1Gsv1 polypeptide.

11. The method of claim 10, wherein said CACNA1Gsv1 polypeptide is obtained by expression of said polypeptide from an expression vector comprising a polynucleotide encoding SEQ ID NO 5.

12. The method of claim 11, wherein said polypeptide consists of SEQ ID NO 5.

13. A method for screening for a compound able to bind to or interact with a CACNA1Gsv1 protein or a fragment thereof comprising the steps of:
(a) expressing a CACNA1Gsv1 polypeptide comprising SEQ ID NO 5 or fragment thereof from a recombinant nucleic acid;
(b) providing to said polypeptide a labeled CACNA1G ligand that binds to said polypeptide and a test preparation comprising one or more compounds; and
(c) measuring the effect of said test preparation on binding of said labeled CACNA1G ligand to said polypeptide, wherein a test preparation that alters the binding of said labeled CACNA1G ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell

16. The method of claim 13, wherein said polypeptide is expressed from an expression vector

17. The method of claim 13, wherein said CACNA1Gsv1 ligand is a CACNA1G inhibitor.

18. The method of claim 16, wherein said expression vector comprises SEQ ID NO 4 or a fragment of SEQ ID NO 4.

19. The method of claim 16, wherein said polypeptide comprises SEQ ID NO 5 or a fragment of SEQ ID NO 5.

20. A method of screening for CACNA1Gsv1 activity comprising the steps of:
(a) contacting a cell expressing a recombinant nucleic acid encoding CACNA1Gsv1 comprising SEQ ID NO 5 with a test preparation comprising one or more test compounds; and
(b) measuring the effect of said test preparation on ion channel activity.
